(19) United States
(12) Patent Application Publication
(10) Pub. No.: US 2009/0104207 A1
(43) Pub. Date: Apr. 23, 2009

(54) Hepatitis C Virus Neutralizing Antibodies

(76) Inventors: Erik Depla, Destelbergen (BE); Robert Purcell, Gaithersburg, MD (US); Jens Bukh, Kensington, MD (US); Suzanne Emerson, Gaithersburg, MD (US); Jens-Christophe Meunier, Bethesda, MD (US)

Correspondence Address: NIXON & VANDERHYE, PC, 901 NORTH GLEBE ROAD, 11TH FLOOR, ARLINGTON, VA, 22203, US

(21) Appl. No.: 12/225,443

(22) PCT Filed: Mar. 22, 2007
(86) PCT No.: PCT/US07/07178

§ 371 Date: Sep. 22, 2008

Related U.S. Application Data
(60) Provisional application No. 60/743,667, filed on Mar. 22, 2006.

(30) Foreign Application Priority Data
Mar. 31, 2006 (EP) 06112063.0

(51) Int. Cl. A61K 39/395; C07K 16/18; C12Q 1/70; C12P 21/04

(52) U.S. Cl. 424/161.1; 530/387.1; 530/388.3; 530/387.3; 435/5; 435/69.6

(57) ABSTRACT

The invention relates to anti-HCV antibodies and more specifically to neutralizing anti-HCV antibodies and their variable and complementarity determining regions (CDR). In particular, the neutralizing anti-HCV antibodies are neutralizing anti-HCV envelope protein 1 (HCV E1) antibodies. Also subject of the invention are compositions comprising these antibodies, CDRs or variable regions, and compounds comprising at least one of the CDRs or variable regions of said antibodies. Further subject of the invention are the application of any of said antibodies, CDRs, variable regions or compounds in HCV prophylaxis, therapy, and diagnosis, as well as methods for producing the antibodies.

This application claims benefit of U.S. Provisional Patent Application No. 60/743,667, filed Mar. 22, 2006 and EP 06 112 063.0, filed Mar. 31, 2006, the entire contents of each of which is incorporated herein by reference.

This invention was created in the performance of a Cooperative Research and Development Agreement with the National Institutes of Health, an Agency of the Department of Health and Human Services. The Government of the United States has certain rights in this invention.

### FIELD OF THE INVENTION

The invention relates to anti-HCV antibodies and more specifically to neutralizing anti-HCV antibodies and their variable and complementarity determining regions (CDR). In particular, the neutralizing anti-HCV antibodies are neutralizing anti-HCV envelope protein 1 (HCV E1) antibodies. Also subject of the invention are compositions comprising these antibodies, CDRs or variable regions, and compounds comprising at least one of the CDRs or variable regions of said antibodies. Further subjects of the invention are the application of any of said antibodies, CDRs, variable regions or compounds in HCV prophylaxis, therapy, and diagnosis, as well as methods for producing the antibodies.

### BACKGROUND OF THE INVENTION

The about 9.6 kb single-stranded RNA genome of the HCV virus comprises a 5′- and 3′-non-coding region (NCRs) and, in between these NCRs a single long open reading frame of about 9 kb encoding an HCV polyprotein of about 3000 amino acids.

HCV polypeptides are produced by translation from the open reading frame and cotranslational proteolytic processing. Structural proteins are derived from the amino-terminal one-fourth of the coding region and include the capsid or Core protein (about 21 kDa), the E1 envelope glycoprotein (about 35 kDa) and the E2 envelope glycoprotein (about 70 kDa, previously called NS1), and p7 (about 7 kDa). The E2 protein can occur with or without a C-terminal fusion of the p7 protein (Shimotohno et al. 1995). Recently, an alternative open reading frame in the Core-region was found which is encoding and expressing a protein of about 17 kDa called F (Frameshift) protein (Xu et al. 2001; Ou & Xu in US Patent Application Publication No. US2002/0076415). In the same region, ORFs for other 14-17 kDa ARFPs (Alternative Reading Frame Proteins), A1 to A4, were discovered and antibodies to at least A1, A2 and A3 were detected in sera of chronically infected patients (Walewski et al. 2001). From the remainder of the HCV coding region, the non-structural HCV proteins are derived which include NS2 (about 23 kDa), NS3 (about 70 kDa), NS4A (about 8 kDa), NS4B (about 27 kDa), NS5A (about 58 kDa) and NS5B (about 68 kDa) (Grakoui et al. 1993).

HCV is the major cause of non-A, non-B hepatitis worldwide. Acute infection with HCV (20% of all acute hepatitis infections) frequently leads to chronic hepatitis (70% of all chronic hepatitis cases) and end-stage cirrhosis. It is estimated that up to 20% of HCV chronic carriers may develop cirrhosis over a time period of about 20 years and that of those with cirrhosis between 1 to 4%/year is at risk to develop liver carcinoma (Lauer & Walker 2001, Shiffman 1999). An option to increase the life-span of HCV-caused end-stage liver disease is liver transplantation (30% of all liver transplantations world-wide are due to HCV-infection).

The FDA-approved options for treating HCV infection are very limited and normally comprise a treatment regimen of ribavirin and interferon-alfa (or pegylated interferon-alfa). Even the most optimal treatment regimen today (combination of pegylated interferon-alfa with ribavirin and with extension of the therapy based on genotype and viral load) results in severe side effects (about 10% of patients have to discontinue because of side effects, and overall about 25% of patients stop therapy prematurely), and of those able to complete the treatment schedule only 42-46% show a sustained response if they are infected with genotype 1, the most predominant genotype world-wide (Manns et al. 2001). In addition, this therapy is not advised for patients with pre-existing markers of anemia, auto-immune diseases or a history of depression which are already frequent conditions in HCV. Because of these and other medical complications, up to 75% of the HCV patients are excluded from therapy today (Falck-Ytter et al. 2002).

In view of the paucity of available treatments, many different compounds are currently being evaluated in clinical trials for their efficacy in treating and/or preventing the development of disease symptoms associated with HCV infection. Prevention of HCV infection therein is generally accepted to refer to prevention of chronic HCV infection as all data available today point at the near impossibility to establish sterilising immunity, i.e., acute HCV infection cannot be prevented. The compounds under evaluation comprise anti-phospholipid therapy with Tarvacin (Peregrine Pharmaceuticals Inc), other interferons (Amarillo Biosciences; Flamel Technologies; Human Genome Sciences; BioMedicine; Ares-Serono; InterMune), polymerase inhibitors (ViroPharma/Wyeth; AKROS Pharma; Idenix Pharmaceuticals), vaccines (Chiron; Intercell; Innogenetics), serine proteases (Schering; Boehringer-Ingelheim), isatoribine or modified forms thereof (ANADYS), protease inhibitors (Schering; Vertex), antisense compounds (BioPharma; Isis Pharmaceutical/Elan), immunomodulators (Coley; SciClone), caspase inhibitors (Idun Pharmaceuticals), histamine (Maxim), antivirals (Bioenvision; Endo Labs Solvay), glucosidase I inhibitors (MIGENIX), anti-fibrotics (Indevus), and nucleoside analogues (Valeant Pharmaceuticals).

Further under clinical evaluation are a number of antibodies:
an anti-CD20 monoclonal antibody known as Rituximab or Rituxam (Genentech/IDEC) for treatment of cryoglobulinemia, one of the symptoms of HCV infection;
a mixture of 2 anti-HCV E2 monoclonal antibodies, administered to patients during and after liver transplantation in order to prevent post-transplant recurrence of HCV;
a polyclonal antibody preparation known as Civacir (NABI), administered to patients during and after liver transplantation in order to prevent post-transplant recurrence of HCV.

Other antibodies known in the art as being neutralizing HCV antibodies or potentially neutralizing HCV antibodies are disclosed by:
Rosa et al. 1996: assay to quantify E2 binding to target cells, and to quantify neutralization of this binding (also termed NOB or neutralization of binding); assay applied to polyclonal HCV antibodies from chimpanzee sera;
Shimizu et al. 1994: assay to assess neutralization of HCV infection using HCV-carrying serum to infect HPB-Ma cells; assay applied to polyclonal HCV antibodies;
Li and Allain 2005: two humanized anti-HCV E2 monoclonal antibodies, inhibition of HCV binding to Molt-4 cells;
Owsianka et al. 2005: anti-HCV E2 HVR1 monoclonal antibody, inhibiting HCV retroviral pseudotype particles (HCVpp) infection of Huh-7 cells, E2 epitope covering amino acids 412 to 423 of the HCV polyprotein;
Keck et al. 2004a: three anti-HCV E2 monoclonal antibodies, inhibiting HCV retroviral pseudotype particles (HCVpp) attachment and entry to Huh-7 cells, E2 epitopes are conformational and do not involve HVR1 region of E2, all three antibodies inhibit E2-CD81 interaction;
Habersetzer et al. 1998: anti-HCV E2 monoclonal antibody, assay of Rosa et al. 1996, E2 epitope is conformational;
Allander et al. 2000: four anti-HCV E2 monoclonal antibodies inhibiting E2-CD81 interaction, E2 epitopes are conformational and do not involve HVR1 region of E2
Siemoneit et al. 1995: four anti-HCV E1 monoclonal antibodies, no neutralizing activity reported, E1 epitopes located at amino acids 317-322 and 320-326 of the HCV polyprotein (see also Example 5 of the instant invention);
Keck et al. 2004: anti-HCV E1 monoclonal antibody, inhibiting binding of baculovirus-derived HCV-like particles to Molt-4 cells and infection of Raji cells by HCV virions, E1 epitope located at amino acids 192-205 of the HCV polyprotein;
Schofield et al. 2005: anti-HCV E2 monoclonal antibodies, inhibiting HCV retroviral pseudotype particles (HCVpp) infection of Huh-7 cells, E2 epitopes are conformational
Burioni 2005 (US2005/0084845): anti-HCV E2 monoclonal antibodies, inhibiting HCV-VSV infection, E2 epitope are conformational
Maruyama et al 2002 (WO 02/059340): anti-HCV E2 monoclonal antibody, assay of Rosa et al. 1996, E2 epitope is conformational.

A review on HCV neutralizing antibodies is given by Kaplan et al. (2003) and Logvinoff et al. (2004) elaborate on the neutralizing antibody response during acute and chronic HCV infection.

Knowing that of all compounds entering clinical trials only about 10% ultimately passes regulatory approval and reaches the market, there is clearly a continuous need to provide new candidate molecules or compounds for treatment and/or prevention of HCV infection. The need for molecules or compounds for prevention of HCV infection actually is fully unmet as today no single such molecule or compound ended up in an approved drug.

### BRIEF SUMMARY OF THE INVENTION

A first aspect of the invention relates to an isolated anti-HCV E1 envelope protein antibody characterized in that said antibody is capable of neutralizing HCV infection.

In a first embodiment, said neutralizing anti-HCV antibody is further characterized in that it comprises at least one of the complementarity determining region (CDR) amino acid sequences chosen from SEQ ID NOs: 1 to 6 or a CDR with an amino acid sequence that is at least 80% identical with any of SEQ ID NOs: 1 to 6. In an alternative embodiment, the neutralizing anti-HCV antibodies of the invention are characterized in that they comprise a variable region amino acid sequence chosen from SEQ ID NOs: 7 or 8 or an amino acid sequence that is at least 70% identical with any of SEQ ID NOs: 7 or 8.

Another embodiment of the invention defines the neutralizing anti-HCV antibodies by their specificity for binding an HCV E1 envelope protein epitope with SEQ ID NO:17.

As a specific embodiment, the neutralizing anti-HCV antibodies of the invention are human monoclonal antibodies or humanized monoclonal antibodies.

A second aspect of the invention relates to active fragments of the neutralizing anti-HCV antibodies of the invention.

The invention further relates to compositions comprising a neutralizing anti-HCV antibody of the invention and/or an active fragment thereof, and at least one of a carrier, adjuvant, or diluent.

Another aspect of the invention covers diagnostic kits for detecting HCV E1 antigens in a biological sample, said kit comprising a neutralizing anti-HCV antibody or an active fragment thereof as described above.

Methods of producing the above-described neutralizing anti-HCV antibodies, or active fragments thereof, form an integral aspect of the invention. In particular, such methods can comprise the steps of:
(i) obtaining a crude preparation of said antibody or antibody fragment by means of recombinant expression of the antibody or antibody fragment, or by means of chemical synthesis of the antibody or antibody fragment;
(ii) purifying said antibody or antibody fragment from the crude preparation obtained in (i).

Alternatively, an active fragment of the neutralizing anti-HCV antibodies of the invention can be obtained or produced by a method comprising the steps of:
(i) obtaining a crude preparation of an antibody comprising said fragment by means of recombinant expression of the antibody or by means of chemical synthesis of the antibody;
(ii) purifying said antibody from the crude preparation obtained in (i).
(iii) isolating the active fragment from the antibody purified in (ii).

The neutralizing anti-HCV antibodies of the invention, or the active fragments thereof, are useful in many applications for preventing or treating HCV infection. Several embodiments of this aspect are summarized hereafter as uses of the neutralizing anti-HCV antibodies of the invention, or active fragments thereof, in:
passive immunization of a healthy or HCV infected mammal;
prevention of HCV recurrence in a non-HCV infected liver transplanted to a chronic HCV patient;
prevention of HCV infection in a non-HCV infected mammal;
prevention of HCV infection in a non-HCV infected mammal after an accident with an HCV-bearing needle-stick;
prevention of transmission of HCV infection during pregnancy and/or birth from an HCV infected mother mammal to its child;
treatment of HCV infection in an HCV infected mammal.

In any of the above uses, the neutralizing anti-HCV antibodies of the invention, or the active fragments thereof, can be further combined with any other anti-HCV medicament wherein said combination occurs prior to, simultaneously with or after said other anti-HCV medicament. Alternatively, in any of the above uses, the neutralizing anti-HCV antibodies of the invention, or the active fragments thereof, can be further combined with any other HCV therapy wherein said combination occurs prior to, simultaneously with or after said other HCV therapy. In the above, mammals clearly include humans.

The invention further relates to in vitro methods for identifying compounds capable of neutralizing HCV infection, said methods including the steps of:
(i) setting up an assay allowing the neutralizing anti-HCV antibody of the invention, or an active fragment thereof, to interact with E1, or with parts of E1 comprising SEQ ID NO:17,
(ii) adding the compound to be assessed for HCV neutralizing activity prior to, concurrently with, or after contacting the antibody with E1 or parts of E1 as in (i),
(iii) reading out the binding of the antibody with said E1 or parts of E1,
(iv) identifying, from (iii), whether or not the compound added in (ii) qualifies as a compound capable of interfering with the antibody-E1 interaction
(v) confirming the neutralizing activity of the compound identified in (iv) in an HCV neutralization assay.

Another aspect of the invention relates to methods for determining the neutralizing activity of a compound on HCV infection, said methods including the use of the above-described neutralizing anti-HCV antibodies, or the active fragments thereof, as a positive control compound for neutralization of HCV infection.

The invention further relates to an isolated complementarity determining region (CDR) of an anti-HCV E1 envelope protein antibody capable of neutralizing HCV infection. In one embodiment thereto, said CDR has an amino acid sequences chosen from SEQ ID NOs: 1 to 6 or a CDR with an amino acid sequence that is at least 80% identical with any of SEQ ID NOs: 1 to 6. Alternatively, said CDR is encoded by a nucleic acid sequence chosen from SEQ ID NOs: 9 to 14. Said CDR can also be incorporated in a composition further comprising for instance a carrier, adjuvant, or diluent.

The invention also relates to an isolated variable region of an anti-HCV E1 envelope protein antibody capable of neutralizing HCV infection. In one embodiment thereto, said variable region has an amino acid sequence which is chosen from SEQ ID NOs: 7 or 8 or an amino acid sequence that is at least 70% identical with any of SEQ ID NOs: 7 or 8. Alternatively, said variable region is encoded by a nucleic acid sequence chosen from SEQ ID NOs: 15 or 16. Said variable region can also be incorporated in a composition further comprising for instance a carrier, adjuvant, or diluent.

A further aspect of the invention relates to compounds capable of neutralizing HCV infection with said compounds comprising at least one CDR as described above or at least one variable region as described above. Such a compound can be used in passive immunization of a healthy or HCV infected mammal. Clearly, said passive immunization can be combined with any other HCV therapy or any other anti-HCV medicament, and wherein said combination occurs prior to, simultaneously with, or after said other HCV therapy or said other anti-HCV medicament.

Also, such a compound is applicable in methods for determining the neutralizing activity of a compound on HCV infection, said methods including use of said compound as a positive control compound for neutralization of HCV infection. Said compounds can also be incorporated in a composition further comprising for instance a carrier, adjuvant, or diluent.

The invention further relates to in vitro methods for identifying compounds capable of neutralizing HCV infection, said methods including the steps of:
(i) setting up an assay allowing an isolated CDR or isolated variable region as described above, or a compound comprising at least one CDR or at least one variable region as described above, to interact with E1, or with parts of E1 comprising SEQ ID NO:17,
(ii) adding the compound to be assessed for HCV neutralizing activity prior to, concurrently with, or after contacting an isolated CDR or isolated variable region as described above, or a compound comprising at least one CDR or at least one variable region as described above with E1 or parts of E1 as in as in (i),
(iii) reading out the binding of an isolated CDR or isolated variable region as described above, or a compound comprising at least one CDR or at least one variable region as described above, with said E1 or parts of E1,
(iv) identifying, from (iii), whether or not the compound added in (ii) qualifies as a compound capable of interfering with the interaction between an isolated CDR or isolated variable region as described above, or a compound comprising at least one CDR or at least one variable region as described above, and said E1 or parts of E1,
(v) confirming the neutralizing activity of the compound identified in (iv) in an HCV neutralization assay.

### DESCRIPTION OF THE DRAWINGS

FIG. 1. Neutralization observed in a preliminary screening of 14 antibodies specific to E1. Antibodies have been tested at a concentration of 50 μg/ml and are identified as neutralizing if at least 50% neutralization versus control is observed. See Example 3 for technical details.

FIG. 2 (2A-2F). Alignment of the epitope amino acids 313-326 of E1 performed on the HCV Los Alamos database (http://hcv.lanl.gov/content/hcv-db/index) on 5 Jan. 2006.

FIG. 3. The heavy chain specific consensus amino acid sequence for neutralizing antibody 5D2. Theoretically predicted CDR loops are underlined (based on consensus sequence rules). CDR amino acid sequences of the neutralizing anti-HCV antibody are further defined by the bold underlined SEQ ID NOs: 1 to 3; the heavy chain variable regions of the neutralizing anti-HCV antibody is defined by SEQ ID NO: 7.

FIG. 4. The light chain specific consensus amino acid sequence for neutralizing antibody 5D2. Theoretically predicted CDR loops are underlined (based on consensus sequence rules). CDR amino acid sequences of the neutralizing anti-HCV antibody are further defined by the bold underlined SEQ ID NOs: 4 to 6; the heavy chain variable regions of the neutralizing anti-HCV antibody is defined by SEQ ID NO: 8.

FIG. 5. Nucleic acid sequence of the variable region of the heavy chain (VH) of the neutralizing anti-HCV antibody (5D2) with in bold and underlined the CDR-encoding sequences (CDR-H1, CDR-H2 and CDR-H3).

FIG. 6. Nucleic acid sequence of the variable region of the light chain (VL) of the neutralizing anti-HCV antibody (5D2) with in bold and underlined the CDR-encoding sequences (CDR-L1, CDR-L2 and CDR-L3).

FIG. 7. Effect of Ala (or Gly)-substitutions on binding of neutralizing anti-HCV antibody 5D2 to the epitope. The difference in log EC50 versus IGP 2254 for each of the alanine (glycine) variants is shown. A positive delta log EC50 indicates a reduced binding. A negative delta log EC50 indicates an increased binding.

FIG. 8. Effect of natural sequence variation on binding of neutralizing anti-HCV antibody 5D2 to the epitope. The difference in log EC50 versus IGP 2254 for each of the natural sequence variants is shown. A positive delta log EC50 indicates a reduced binding. A negative delta log EC50 indicates an increased binding. The “>” on top of the bar corresponding to the peptide with sequence “GHRMAWDMM” means “more than” the value indicated by the bar.

FIG. 9. ELISA results of binding of neutralizing anti-HCV antibody 5D2 at 1.25 μg of antibody/mL to biotinylated E1 peptides presented on streptavidin coated microtiterplates.

### DETAILED DESCRIPTION OF THE INVENTION

The current invention contributes to the quest for candidate molecules for treatment and/or curing and/or prevention of HCV infection. As the candidate molecules are anti-HCV antibodies, they can also be applied for diagnosing HCV infection. Unexpectedly, the anti-HCV antibodies of the invention are capable of neutralizing HCV infection. This feature distinguishes these anti-HCV antibodies, or more precisely, these neutralizing anti-HCV antibodies from the anti-HCV antibodies known in the art that are not neutralizing. In particular the neutralizing anti-HCV antibodies of the invention recognize an epitope in the HCV envelope protein 1 (HCV E1) and hence are HCV neutralizing anti-HCV E1 antibodies.

Specifically, the (human) neutralizing anti-HCV (monoclonal) antibodies of the invention have been obtained as described in Example 2 herein. The neutralizing activity of these antibodies was determined as described in Example 3 (neutralization of HCV type 1a in a HCV pp system, see further), in Examples 5 and 6 (neutralization of HCV types 1 to 6 in a HCV pp system, see further); in the initial neutralization assays murine monoclonal antibodies (Example 1) binding to a similar epitope were incorporated. The neutralizing anti-HCV antibodies were further characterized in terms of their amino acid- and nucleic acid sequences (Example 7) and in terms of their epitope (Examples 4 and 9-11). The affinity of the neutralizing anti-HCV antibodies for their HCV E1 epitope was determined in Example 8.

Therefore, a first aspect of the invention relates to an isolated anti-HCV E1 envelope protein antibody characterized in that said antibody is capable of neutralizing HCV infection.

“Neutralization” of viruses, in particular HCV, is defined here as the abrogation of virus infectivity in vitro by the binding of a neutralizing compound to the virion. Thus, the target of the neutralizing compound does not have to be of virus origin, as long as it is present on the virion. The definition does not include the block of infection by a neutralizing compound that binds to a receptor for the virus on the (host) cell surface. It is reasonable to add a further criterion: that neutralizing compounds act before the first major biosynthetic event in the virus replicative cycle has taken place. Then, it is a matter for experimental investigation whether neutralization can block a step between virus entry and that later event. According to this criterion, interference with release of progeny virus should not be termed neutralization (adapted from Klasse and Sattentau, 2002).

This definition excludes any compounds to be defined as neutralizing which only inhibit binding of HCV or HCV like particles or isolated HCV envelope proteins to its candidate receptors (such as CD81, SRB-I, LDL-receptor) unless such compounds would also abrogate or block virus infectivity. To assess neutralization of HCV in vitro, a few assays currently qualify. These neutralization assays include (i) the pseudoparticle assays as initially described by Bartosch et al (2003) and Hsu (2003) as these assays use the entire E1 and E2 sequence as part of a pseudotype particle to study infectivity; and (ii) the HCV in vitro cell culture systems available since 2005 (for review, see Berke and Moradpour 2005). The pseudotype assays generally rely on retroviral/lentiviral core viral particles displaying unmodified functional HCV envelope proteins. The core viral particles herein can be, e.g., HIV or MLV. Infectivity of the pseudotype particles, e.g., HIV-HCVpp or MLV-HCVpp, is usually measured via the expression of a reporter gene such as luciferase or GFP. It is meanwhile generally recognized that these assays are convenient and robust (see, e.g., Berke and Moradpour 2005). It is further accepted that, in order to qualify as truly neutralizing, a compound should display a neutralizing activity of 50% or more in one of the above pseudotyped viral particle assays or in the in vitro cell culture systems (see Bartosch et al., 2003a,b; Hsu et al. 2003; Lindenbach et al. 2005; Wakita et al. 2005).

Assays such as the ones initially described by Lagging et al (1998) do not qualify as they use E1 and E2 sequences of which the transmembrane domains have been substituted for the one of VSV-G protein. The latter assay can not guarantee that the entire entry process is mediated by E1 and/or E2 of HCV. Moreover, in such pseudotype particles the E1/E2 presentation is expected to be different from pseudotype particles in which the E1/E2 is completely present. Infectivity of VSV-HCV pseudotype viruses is measured via plaque formation, i.e., by determination of pseudotype PFU (plaque-forming units) titer. The validity of results obtained with the VSV-HCVpp test has been questioned (see, e.g., Buonocore et al. 2002). Other tests that do not qualify include the “NOB” assay (NOB=neutralization of binding) and the assay based on baculovirus-expressed HCV-like particles (HCV-LP). The NOB assay only assesses the binding of purified go recombinant E2 protein to susceptible target cells (Rosa et al. 1996). It is generally accepted that no proven correlation exists between NOB activity and true virus neutralizing activity of a compound (see, e.g., Burioni et al. 1998, page 813, right-hand column). The HCV-LPs are produced in insect cells by baculovirus expressing HCV core, E1, E2, p7, and part of NS2. Although dye-labeled HCV-LPs can be internalized into the cytoplasm of susceptible cells (several hepatic cell lines, but also a T-cell line), this assay is mainly suited for assessing attachment of HCV-LP to such cells (Triyatni et al. 2002). Drawbacks of HCV-LPs include a glycosylation of the HCV envelope proteins that is different from that of HCV envelope proteins produced in mammalian cells.

Elaborating on the first aspect of the invention which relates to isolated HCV-neutralizing anti-HCV E1 envelope protein antibodies, this relates to said antibodies whose neutralizing activity is established/determined in an assay for determining the capacity to neutralize HCV pseudotype particles.

In one embodiment, said neutralizing capacity is determined by measuring the activity of a reporter gene product (e.g., luciferase, GFP). A further criterion that may be, but not necessarily must be, included is that said HCV-neutralizing anti-HCV E1 envelope protein antibodies should, in said suitable assay, display a neutralizing capacity of at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%; and this at a concentration of the antibody in the assay of no more than 50 μg/mL, no more no more than 40 μg/mL, no more than 30 μg/mL, no more than 25 μg/mL, no more than 20 μg/mL, no more than 15 μg/mL, no more than 10 μg/mL, no more than 5 μg/mL, no more than 2 μg/mL or no more than 1 μg/mL.

In an alternative embodiment, said neutralizing capacity is determined in a HCV cell culture system. A further criterion that may be, but not necessarily must be, included is that said HCV-neutralizing anti-HCV E1 envelope protein antibodies should, in said suitable assay, display a neutralizing capacity of at least 75%, e.g., at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%; and this at a concentration of the antibody of 100 μg/mL.

The term “antigen” refers to a structure, often a polypeptide or protein, for which an immunoglobulin, such as an antibody, has affinity and specificity.

The terms “antigenic determinant”, “antigenic target” and “epitope” all refer to a specific binding site on an antigen or on an antigenic structure for which an immunoglobulin, such as an antibody, has specificity and affinity.

The term “antibody” refers to a protein or polypeptide having affinity for an antigen or for an antigenic determinant. Such an antibody is commonly composed of 4 chains, 2 heavy- and 2 light chains, and is thus tetrameric. An exception thereto are camel antibodies that are composed of heavy chain dimers and are devoid of light chains, but nevertheless have an extensive antigen-binding repertoire. An antibody usually has both variable and constant regions whereby the variable regions are mostly responsible for determining the specificity of the antibody and will comprise complementarity determining regions (CDRs).

The term “specificity” refers to the ability of an immunoglobulin, such as an antibody, to bind preferentially to one antigenic target versus a different antigenic target and does not necessarily imply high affinity.

The term “affinity” refers to the degree to which an immunoglobulin, such as an antibody, binds to an antigen so as to shift the equilibrium of antigen and antibody toward the presence of a complex formed by their binding. Thus, where an antigen and antibody are combined in relatively equal concentration, an antibody of high affinity will bind to the available antigen so as to shift the equilibrium toward high concentration of the resulting complex.

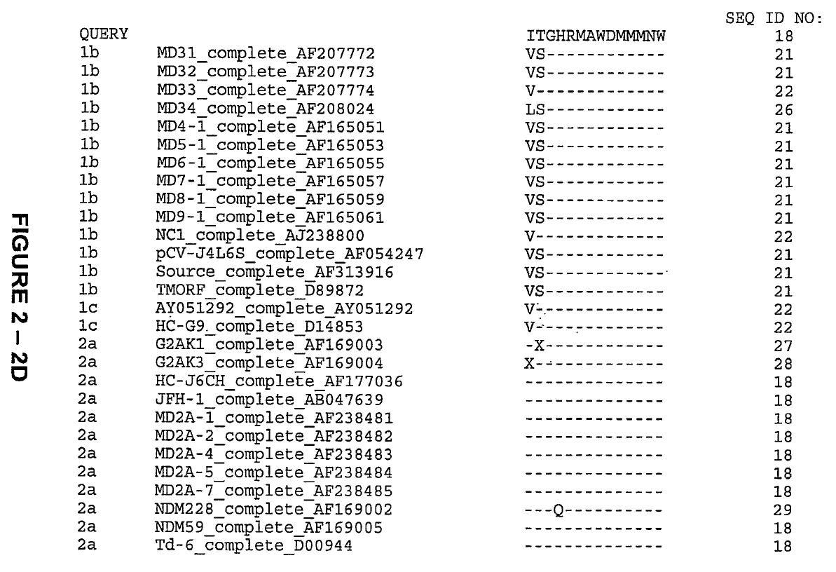

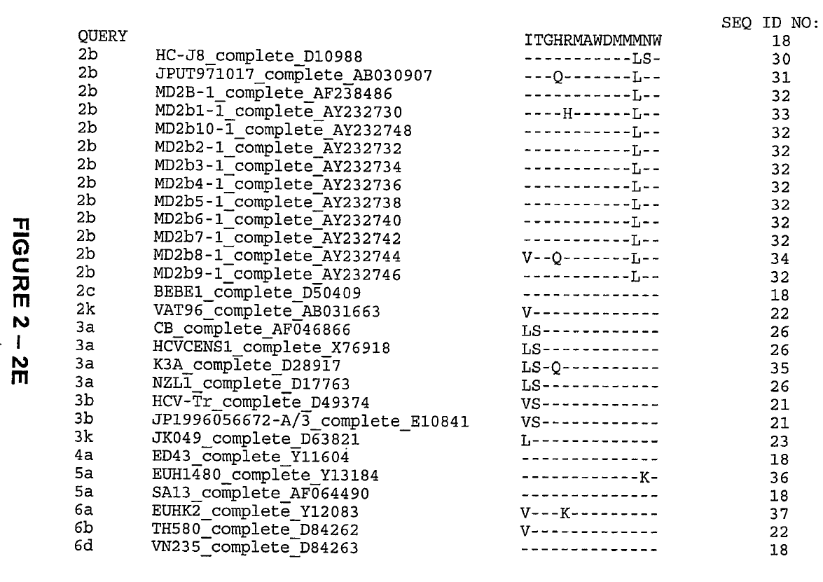

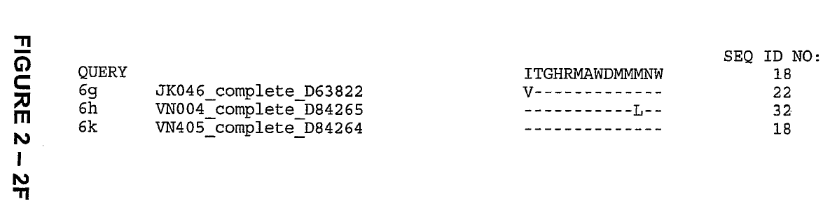

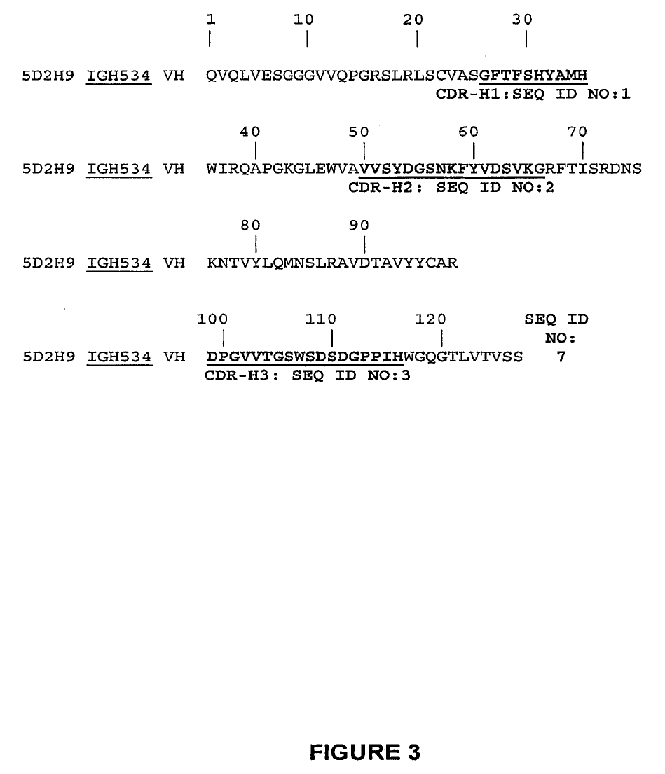

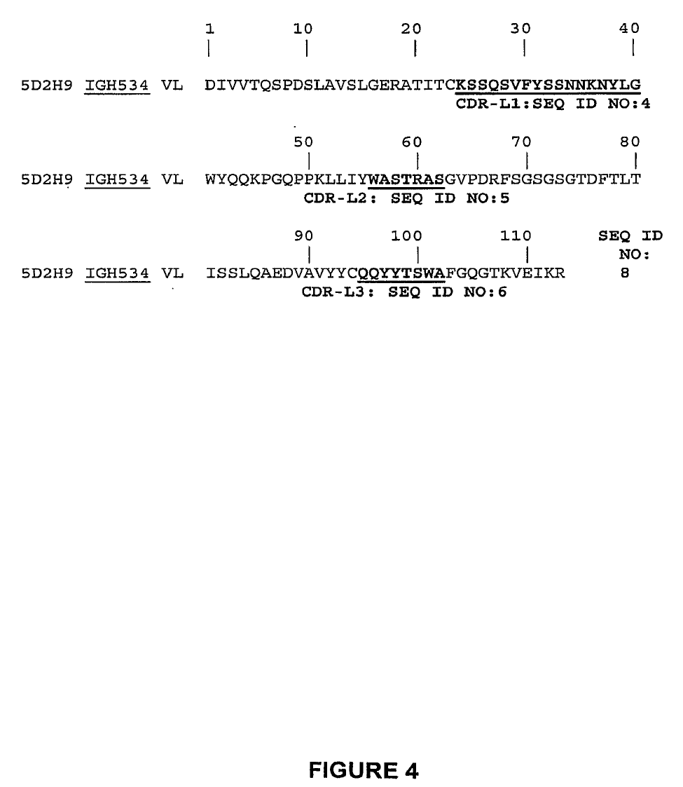

The term “complementarity determining region” or “CDR” refers to variable regions of either H (heavy) or L (light) chains (also abbreviated as VH and VL, respectively) and contains the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains. The accepted CDR regions have been described by Kabat et al. (1991). In a first embodiment, the neutralizing anti-HCV antibodies of the invention are further characterized in that it comprises at least one of the complementarity determining region (CDR) amino acid sequences chosen from SEQ ID NOs: 1 to 6 or a CDR with an amino acid sequence that is at least 80, 81, 82, 83, 84, 85, 86, 87, 88, 89, 90, 91, 92, 93, 94, 95, 96, 97, 98, or 99% identical with any of SEQ ID NOs: 1 to 6. In an alternative embodiment, the neutralizing anti-HCV antibodies of the invention are characterized in that they comprise a variable region amino acid sequence chosen from SEQ ID NOs: 7 or 8 or with an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical with any of SEQ ID NOs: 7 or 8. The CDR amino acid sequences SEQ ID NOs: 1 to 6, as well as the variable region amino acid sequences SEQ ID NOs: 7 or 8 are depicted in FIGS. 3 and 4 (see also “Description of the drawings”).

A further embodiment of the invention relates to the above described anti-HCV antibodies further characterized in that they comprise
a CDR triplet H1/H2/H3 or a CDR triplet that is at least 85% identical therewith; or
a CDR triplet L1/L2/L3 or a CDR triplet that is at least 85% identical therewith;
   wherein H1 has the amino acid sequence of SEQ ID NOs: 1, H2 has the amino acid sequence of SEQ ID NOs: 2, H3 has the amino acid sequence of SEQ ID NOs: 3, L1 has the amino acid to sequence of SEQ ID NOs: 4, L2 has the amino acid sequence of SEQ ID NOs: 5, and L3 has the amino acid sequence of SEQ ID NOs: 6.

The indication of “CDR triplet” herein refers to the combination of CDR regions of a heavy chain (H1, H2 or H3) or of a light chain (L1, L2 or L3) of an antibody of the invention. In particular, the combination can be a non-contiguous combination such as a combination in an antibody. The order of the individual CDR region in the non-contiguous combination can be at random, e.g., H1/H2/H3, H31H1/H2, H2/H3/H1, etc. The % identity is to be calculated as in the following example. If, for example for a CDR triplet L1/L2/L3, within the L1 region at least 15 out of the 17 amino acids are identical, within the L2 region at least 5 out of the 7 amino acids, and within the L3 region at least 6 out of the 8 amino acids, then in total there should be at least 15 (L1)+5 (L2)+6 (L3)=26 amino acids identical within the total of 17 (L1)+7 (L2)+8 (L3) 32 amino acids. An identity of 26 amino acids on 32 equals 81.25% identity.

Another embodiment of the invention defines the neutralizing anti-HCV antibodies by their specificity for binding an HCV E1 envelope protein epitope with SEQ ID NO: 17. Alternatively, said epitope has the amino acid sequence of SEQ ID NO:18 or an amino acid sequence that is 75, 76, 77, 78, 79, 80, 81, 82, 83, 84, 85, 86, 87, 88, 89, 90, 91, 92, 93, 94, 95, 96, 97, 98, or 99% identical with SEQ ID NO:18

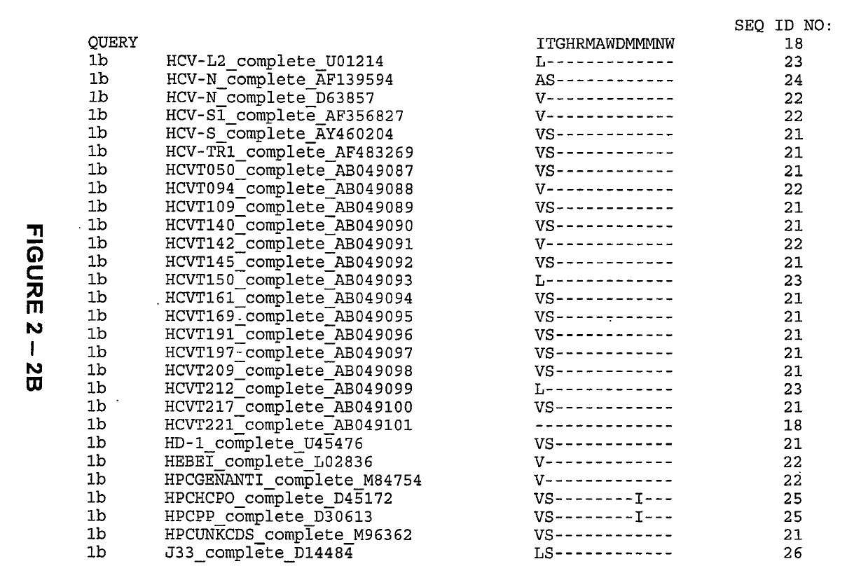

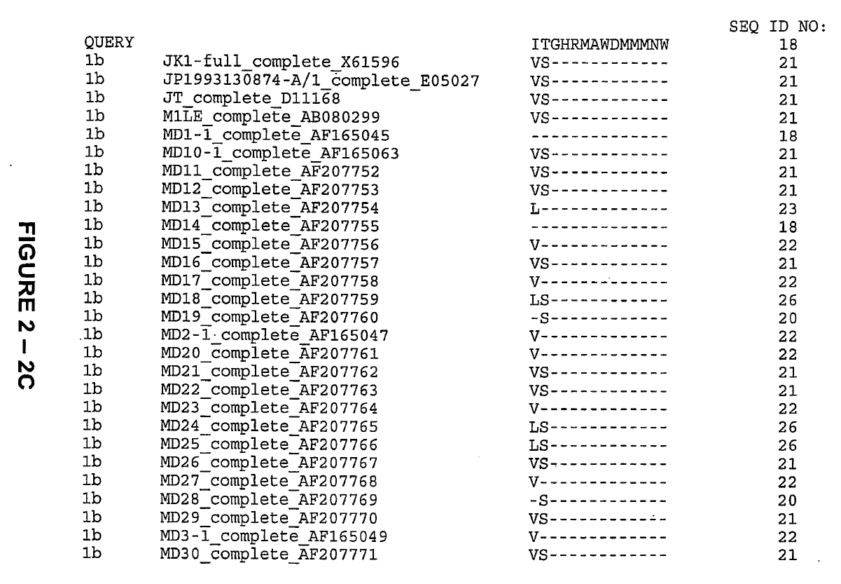

The E1 epitope of the neutralizing anti-HCV antibodies of the invention was delineated as outlined in Example 4, and its location was determined to E1 amino acids 313 to 326 (amino acid numbering relative to the HCV polyprotein). As the neutralizing anti-HCV antibodies of the invention are capable of neutralizing infection by most of the known HCV genotypes (types 1 to 6), the epitope sequence is not fully constrained and allows the presence of HCV genotype-specific amino acid variations. SEQ ID NO:17 constitutes the consensus epitope sequence for HCV types 1 to 6 as derived from FIG. 2 and has the formula:

| | |
|---|---|
| X1-X2-G-X3-X4-MAW-X5-M-X6-X7-X8-W | (SEQ ID NO:17) |

wherein
X1 is I, V, L or A;
X2 is T or S;
X3 is H or Q;
X4 is R, H or K;
X5 is D or N;
X6 is M or I;
X7 is M or L; and
X8 is N, S or K.

Alternatively, FIG. 2 allows the defining of the E1 epitope of the neutralizing anti-HCV antibodies of the invention as SEQ ID NO:18 (E1 amino acids 313 to 326 of an HCV genotype 1a isolate) or any E1 epitope sequence that is at least 75, 76, 77, 78, 79, 80, 81, 82, 83, 84, 85, 86, 87, 88, 89, 90, 91, 92, 93, 94, 95, 96, 97, 98, or 99% identical with SEQ ID NO:18.

SEQ ID NO: 18 has the formula: ITGHRMAWDMMMNW (see FIG. 2).

As supported by Examples 9 and 10, binding of the neutralizing anti-HCV E1 envelope protein antibodies of the invention to their epitopes is insensitive to naturally occurring sequence variation within the epitope, as well as insensitive to replacement/substitution of epitope amino acids by alanine or glycine. Thus, any of the epitope variants described above are to be considered as “immunologically active variants” all capable of binding the neutralizing anti-HCV antibodies of the invention. Such equivalents thus include strain, subtype (=genotype), or type(group)-specific variants, e.g. of the currently known sequences or strains belonging to genotypes 1 to 6 (and subtypes thereof); see Simmonds et al. 2005.

Further fine-mapping of the epitopes recognized by the neutralizing anti-HCV E1 envelope protein antibodies confirms that the common epitope is defined by the E1 region spanning amino acids 313-326. In yet further detail, the said anti-HCV antibodies are not binding to any of SEQ ID NOs: 46, 47, or 49.

The neutralizing anti-HCV E1 envelope protein antibodies of the invention are further characterized by the binding affinity to their epitope. As such, said antibodies can alternatively be defined by their epitope affinity constant (KD=dissociation constant kd/association constant ka), said affinity constant as measured against IGP 2254 (SEQ ID NO:48) is preferably equal to or lower than 1×10⁻⁹, 7.5×10⁻¹⁰, 5×10⁻¹⁰, 2.5×10⁻¹⁰, 10⁻¹⁰, 7.5×10⁻¹¹, 6×10⁻¹¹, or 5×10⁻¹¹; or is in the range of 10⁻¹⁰ to 10⁻¹¹ M, or 5×10⁻¹¹ to 10⁻¹⁰ M, or more in particular in the range of 5 to 7.5×10⁻¹¹ M.

As a specific embodiment, the neutralizing anti-HCV antibodies of the invention are human monoclonal antibodies or humanized monoclonal antibodies.

Non-human mammalian antibodies or animal antibodies can be humanized (see for instance Winter and Harris 1993). The antibodies or monoclonal antibodies according to the invention may be humanized versions of for instance rodent antibodies or rodent monoclonal antibodies. Humanisation of antibodies entails recombinant DNA technology, and is departing from parts of rodent and/or human genomic DNA sequences coding for H and L chains or from cDNA clones coding for H and L chains. Techniques for humanization of non-human antibodies are known to the skilled person as these form part of the current state of the art.

A second aspect of the invention relates to active fragments of the neutralizing anti-HCV antibodies of the invention.

The term “active fragment” refers to a portion of an antibody that by itself has high affinity for an antigenic determinant, or epitope, and contains one or more CDRs accounting for such specificity. Non-limiting examples include Fab, F(ab)′2, scFv, heavy-light chain dimers, nanobodies, domain antibodies, and single chain structures, such as a complete light chain or complete heavy chain. An additional requirement for “activity” of said fragments in the light of the present invention is that said fragments are capable of neutralizing HCV infection.

The antibodies of the invention, or their active fragments, can be labeled by an appropriate label, said label can for instance be of the enzymatic, calorimetric, chemiluminescent, fluorescent, or radioactive type.

The invention further relates to compositions comprising a neutralizing anti-HCV antibody of the invention and/or an active fragment thereof, and at least one of a carrier, adjuvant, or diluent. In a specific embodiment thereto, said composition is a vaccine composition. Such vaccine composition may be a prophylactic vaccine composition or a therapeutic vaccine composition. In particular the vaccine compositions can be applied for passive immunization. The insensitivity of the neutralizing anti-HCV antibodies of the invention and/or active fragments thereof to epitope sequence variation (as described above) is of interest because it increases the applicability of said antibodies in passive immunization schemes (said antibodies can “tackle” all HCV genotypes) and decreases the chance that HCV viral mutants evolve (due to immune pressure) that can escape from the passive immunization with said antibodies and/or active fragments thereof.

A “carrier”, or “adjuvant”, in particular a “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. Preferably, a pharmaceutically acceptable carrier or adjuvant enhances the immune response elicited by an antigen. Suitable carriers or adjuvantia typically comprise one or more of the compounds included in the following non-exhaustive list:
large slowly metabolized macromolecules such as proteins, polysaccharides, polylactic acids, polyglycolic acids, polymeric amino acids, amino acid copolymers and inactive virus particles;
aluminium hydroxide, aluminium phosphate (see International Patent Application Publication No. WO93/24148), alum (KAl(SO₄)₂.12H₂O), or one of these in combination with 3-0-deacylated monophosphoryl lipid A (see International Patent Application Publication No. WO93/19780);
N-acetyl-muramyl-L-threonyl-D-isoglutamine (see U.S. Pat. No. 4,606,918), N-acetyl-normuramyl-L-alanyl-D-isoglutamine, N-acetylmuramyl-L-alanyl-D-isoglutamyl-L-alanine2-(1′,2′-dipalmitoyl-sn-glycero-3-hydroxyphosphoryloxy) ethylamine;
RIBI (ImmunoChem Research Inc., Hamilton, Mont., USA) which contains monophosphoryl lipid A (i.e., a detoxified endotoxin), trehalose-6,6-dimycolate, and cell wall skeleton (MPL+TDM+CWS) in a 2% squalene/Tween 80 emulsion. Any of the three components MPL, TDM or CWS may also be used alone or combined 2 by 2;
adjuvants such as Stimulon (Cambridge Bioscience, Worcester, Mass., USA), SAF-1 (Syntex);
adjuvants such as combinations between QS21 and 3-de-O-acetylated monophosphoryl lipid A (see International Patent Application Publication No. WO94/00153) which may be further supplemented with an oil-in-water emulsion (see, e.g., International Patent Application Publication Nos. WO95/17210, WO97/01640 and WO9856414) in which the oil-in-water emulsion comprises a metabolisable oil and a saponin, or a metabolisable oil, a saponin, and
a sterol, or which may be further supplemented with a cytokine (see International Patent Application Publication No. WO98/57659);
adjuvants such as MF-59 (Chiron), or poly[di(carboxylatophenoxy) phosphazene] based adjuvants (Virus Research Institute);
blockcopolymer based adjuvants such as Optivax (Vaxcel, Cytrx) or inulin-based adjuvants, such as Algammulin and GammaInulin (Anutech);
Complete or Incomplete Freund's Adjuvant (CFA or IFA, respectively) or Gerbu preparations (Gerbu Biotechnik). It is to be understood that Complete Freund's Adjuvant (CFA) may be used for non-human applications and research purposes as well;
a saponin such as QuilA, a purified saponin such as QS21, QS7 or QS17, β-escin or digitonin;
immunostimulatory oligonucleotides comprising unmethylated CpG dinucleotides such as [purine-purine-CG-pyrimidine-pyrimidine] oligonucleotides. These immunostimulatory oligonucleotides include CpG class A, B, and C molecules (Coley Pharmaceuticals), ISS (Dynavax), Immunomers (Hybridon). Immunostimulatory oligonucleotides may also be combined with cationic peptides as described, e.g., by Riedl et al. (2002);
Immune Stimulating Complexes comprising saponins, for example Quil A (ISCOMS);
excipients and diluents, which are inherently non-toxic and non-therapeutic, such as water, saline, glycerol, ethanol, wetting or emulsifying agents, pH buffering substances, preservatives, and the like;
a biodegradable and/or biocompatible oil such as squalane, squalene, eicosane, tetratetracontane, glycerol, peanut oil, vegetable oil, in a concentration of, e.g., 1 to 10% or 2.5 to 5%;
vitamins such as vitamin C (ascorbic acid or its salts or esters), vitamin E (tocopherol), or vitamin A;
carotenoids, or natural or synthetic flavanoids;
trace elements, such as selenium;
any Toll-like receptor ligand as reviewed in Barton and Medzhitov (2002).

Any of the afore-mentioned adjuvants comprising 3-de-O-acetylated monophosphoryl lipid A, said 3-de-O-acetylated monophosphoryl lipid A may be forming a small particle (see International Patent Application Publication No. WO94/21292).

In any of the aforementioned adjuvants MPL or 3-de-O-acetylated monophosphoryl lipid A can be replaced by a synthetic analogue referred to as RC-529 or by any other amino-alkyl glucosaminide 4-phosphate (Johnson et al. 1999, Persing et al. 2002). Alternatively it can be replaced by other lipid A analogues such as OM-197 (Byl et al. 2003)

More in particular for the antibodies of the invention a “carrier”, or “adjuvant”, or “diluent” in particular a “pharmaceutically acceptable carrier” or “pharmaceutically acceptable adjuvant” or “pharmaceutically acceptable vehicle” is any suitable excipient, diluent, carrier, adjuvant, and/or vehicle which, by themselves, do not induce harmful effects to the individual receiving the composition nor do they elicit protection. Preferably, a pharmaceutically acceptable carrier, adjuvant or vehicle enhances or conserves the activity of the vaccine by buffering, stabilizing, protecting from chemical modification, degradation or aggregation, or controlling the release of the anti-HCV antibody and/or the active fragment thereof. Suitable excipient, diluent, carrier, adjuvant, and/or vehicle typically comprise one or more of the compounds included in the following non-exhaustive list:
large slowly metabolized macromolecules such as proteins, polysaccharides, polylactic acids, polyglycolic acids, polymeric amino acids, amino acid copolymers and poly-ethylene glycols;
excipients and diluents, which are inherently non-toxic and non-therapeutic, such as water, saline, glycerol, ethanol, EDTA, wetting or emulsifying agents, pH buffering substances, preservatives, detergents and more particularly non-ionic detergents such as polysorbate, sugars such as trehalose, sucrose, mannitol, and the like;
vitamins such as vitamin C (ascorbic acid or its salts or esters), vitamin E (tocopherol), or vitamin A;
carotenoids, or natural or synthetic flavanoids;
trace elements, such as selenium.

A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

Typically, a vaccine or vaccine composition is prepared as an injectable, either as a liquid solution or suspension. Injection may be subcutaneous, intramuscular, intravenous, intraperitoneal, intrathecal, intradermal, intraepidermal. Other types of administration comprise implantation, suppositories, oral ingestion, enteric application, inhalation, aerosolization or nasal spray or drops. Solid forms, suitable for dissolving in, or suspension in, liquid vehicles prior to injection may also be prepared. The preparation may also be emulsified or encapsulated in liposomes for enhancing adjuvant effect.

An effective amount of an active substance in a vaccine or vaccine composition is the amount of said substance required and sufficient to elicit an active immune response or the amount of said substance required and sufficient to result in effective passive immunization. It will be clear to the skilled artisan that an active immune response sufficiently broad and vigorous to provoke the effects envisaged by the vaccine composition may require successive (in time) immunizations with the vaccine composition as part of a vaccination scheme or vaccination schedule. Likewise, to provoke the effects envisaged by passive immunization, the vaccine composition may require successive (in time) immunizations with the vaccine composition as part of a vaccination scheme or vaccination schedule. The “effective amount” may vary depending on the health and physical condition of the individual to be treated, the age of the individual to be treated (e.g. dosing for infants may be lower than for adults) the taxonomic group of the individual to be treated (e.g. human, non-human primate, primate, etc.), the capacity of the individual's immune system to mount an effective immune response (in case of active immunization), the degree of protection desired, the formulation of the vaccine, the treating doctor's assessment, the strain and load of the infecting pathogen and other relevant factors. It is expected that the effective amount of the anti-HCV antibodies of the invention will fall in a relatively broad range that can be determined through routine trials. The amount can vary from 0.01 to 1000 μg/dose, more particularly from 0.1 to 100 μg/dose. Usually, however, this amount will vary from 0.1 to 100 mg/kg/dose, more particularly from 0.5 to 20 mg/kg/dose. Dosage treatment may be a single dose schedule or a multiple dose schedule. Dosage may also be adapted such that occurrence of the prozone effect is prevented.

The identification of hitherto unknown and never-identified neutralizing anti-HCV E1 envelope protein antibodies of the invention is a clear incentive to repeat, e.g., the experimental strategy outlined herein (see Examples 2-5 herein) in order to find additional neutralizing anti-HCV E1 envelope protein antibodies. From the art, and from the current invention it is clear that, from all anti-HCV E1 envelope protein antibodies that exist or occur, only a limited subset is actually capable of neutralizing the HCV virus in a thereto suitable assay. By following this procedure, the inventors have identified two additional HCV-neutralizing antibodies, of which one is an anti-HCV E1 envelope protein antibody. This finding clearly underlines, in the context of HCV neutralizing antibodies, the hitherto unrecognized importance of the E1 region and provides a basis to search for additional antibodies as the chance to find neutralizing antibodies is reasonably high.

The inventors thus identified the E1 envelope protein, and more particularly the region represented by SEQ ID NO:17 and SEQ ID NO:18 as a new target in the HCV envelope that can be neutralized by human antibodies. Of the 7 monoclonal antibodies against this target region tested 3 where neutralizing. This finding clearly underlines the importance of this E1 region and provides a basis to search for additional antibodies as the chance to find neutralizing antibodies is high.

An alternative experimental strategy could be the one as followed by, e.g., Farci et al. 1996 who hyperimmunized rabbits with the E2 HVR1 epitope. A polyclonal serum from these rabbits was able to inhibit binding of an E2 protein to susceptible cells (the NOB assay as outlined above). With the suitable and robust HCV neutralization assays available today, the hyperimmunization strategy could be followed using E1 envelope protein (e.g., full-length or overlapping or separate epitopes) and analysing the immune sera for their neutralizing capacity. Techniques to isolate the individual neutralizing monoclonal antibodies from a polyclonal serum are meanwhile well known and form part of the established state of the art.

Another aspect of the invention covers diagnostic kits for detecting HCV E1 antigens in a biological sample, said kit comprising a neutralizing anti-HCV antibody or an active fragment thereof as described above.

Methods of producing the above-described neutralizing anti-HCV antibodies, or active fragments thereof, form an integral aspect of the invention. In particular, such methods can comprise the steps of:
(i) obtaining a crude preparation of said antibody or antibody fragment by means of recombinant expression of the antibody or antibody fragment, or by means of chemical synthesis of the antibody or antibody fragment;
(ii) purifying said antibody or antibody fragment from the crude preparation obtained in (i).

Alternatively, an active fragment of the neutralizing anti-HCV antibodies of the invention can be obtained or produced by a method comprising the steps of:
(i) obtaining a crude preparation of an antibody comprising said fragment by means of so recombinant expression of the antibody or by means of chemical synthesis of the antibody;
(ii) purifying said antibody from the crude preparation obtained in (i).
(iii) isolating the active fragment from the antibody purified in (ii).

In the methods recited above, recombinant expression is not limited to expression in hybridoma cell lines.

The neutralizing anti-HCV antibodies of the invention, or the active fragments thereof, are useful in many applications for preventing or treating HCV infection. Several embodiments of this aspect are summarized hereafter as uses of the neutralizing anti-HCV antibodies of the invention, or active fragments thereof, in:
passive immunization of a healthy or HCV infected mammal;
prevention of HCV recurrence in a non-HCV infected liver transplanted to a chronic HCV patient;
prevention of HCV infection in a non-HCV infected mammal;
prevention of HCV infection in a non-HCV infected mammal after an accident with an HCV-bearing needle-stick;
prevention of transmission of HCV infection during pregnancy and/or birth from an HCV infected mother mammal to its child;
treatment of HCV infection in an HCV infected mammal;
radioimmunotherapy in case the neutralizing anti-HCV antibody, or active fragments thereof, are radiolabelled.

In any of the above uses, the neutralizing anti-HCV antibodies of the invention, or the active fragments thereof, can be further combined with any other anti-HCV medicament wherein said combination occurs prior to, simultaneously with or after said other anti-HCV medicament. Alternatively, in any of the above uses, the neutralizing anti-HCV antibodies of the invention, or the active fragments thereof, can be further combined with any other HCV therapy wherein said combination occurs prior to, simultaneously with or after said other HCV therapy. In the above, mammals clearly include humans.

Another aspect of the invention relates to methods for determining the neutralizing activity of a compound on HCV infection, said methods including the use of the above-described neutralizing anti-HCV antibodies, or the active fragments thereof, as a positive control compound for neutralization of HCV infection.

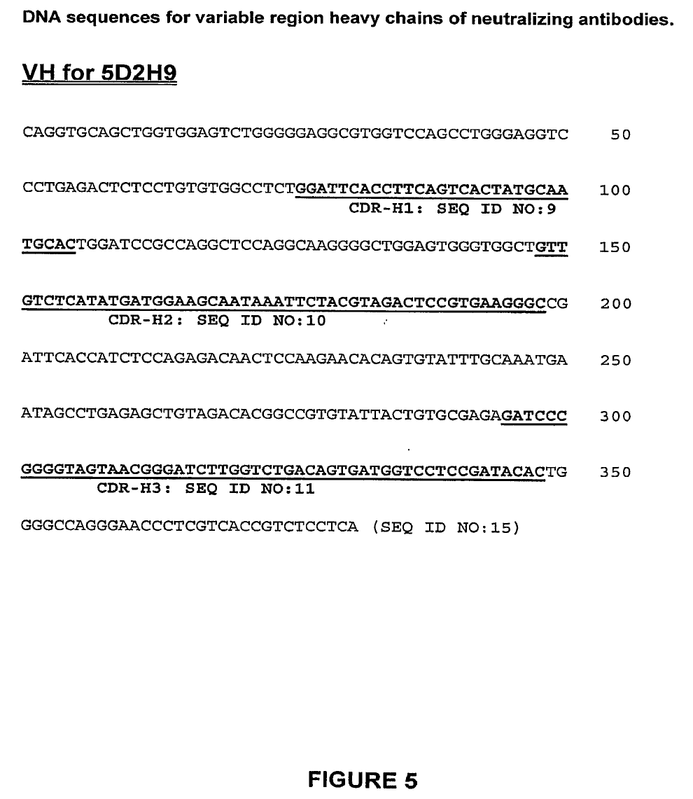

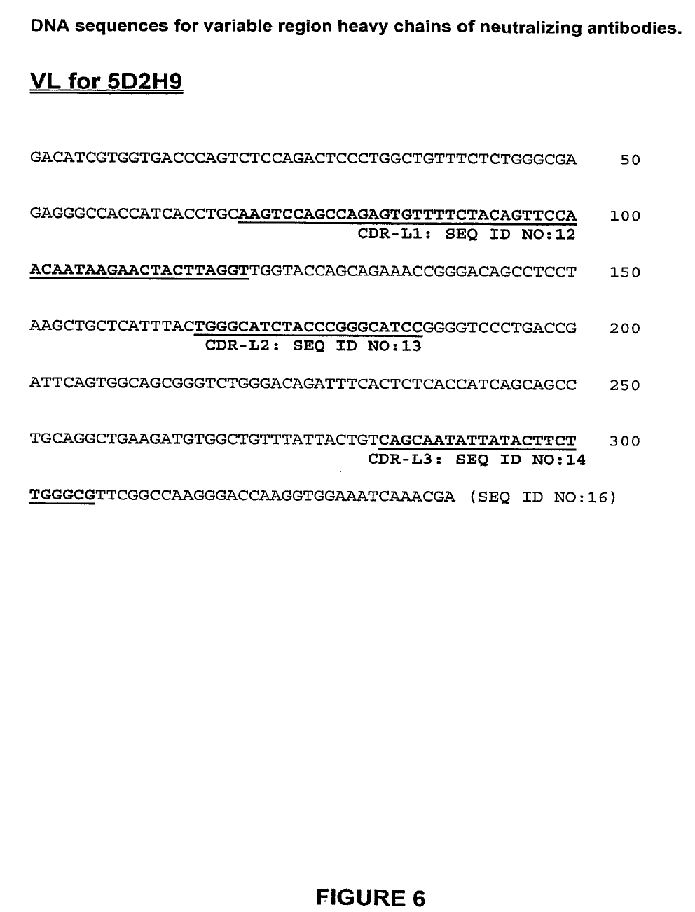

The invention further relates to an isolated complementarity determining region (CDR) of an anti-HCV E1 envelope protein antibody capable of neutralizing HCV infection. In one embodiment thereto, said CDR has an amino acid sequences chosen from SEQ ID NOs: 1 to 6 or a CDR with an amino acid sequence that is at least 80, 81, 82, 83, 84, 85, 86, 87, 88, 89, 90, 91, 92, 93, 94, 95, 96, 97, 98, or 99% identical with any of SEQ ID NOs: 1 to 6. Alternatively, said CDR is encoded by a nucleic acid sequence chosen from SEQ ID NOs: 9 to 14 (see FIGS. 5 and 6). Said CDR can also be incorporated in a composition further comprising for instance a carrier, adjuvant, or diluent. The isolated CDR nucleic acid sequences are part of the invention, as well as any vector or recombinant nucleic acid (DNA, RNA, PNA, LNA, or any hybrid thereof; linear or circular; independent of strandedness) comprising such CDR nucleic acid. Any host cell comprising such CDR nucleic acid sequence, vector or recombinant nucleic acid is likewise part of the invention.

The invention also relates to an isolated variable region of an anti-HCV E1 envelope protein antibody capable of neutralizing HCV infection. In one embodiment thereto, said variable region has an amino acid sequence which is chosen from SEQ ID NOs: 7 or 8 or an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical with any of SEQ ID NOs: 7 or 8. Alternatively, said variable region is encoded by a nucleic acid sequence chosen from SEQ ID NOs: 15 or 16 (see FIGS. 5 and 6). Said variable region can also be incorporated in a composition further comprising for instance a carrier, adjuvant, or diluent. The isolated variable region nucleic acid sequences are part of the invention, as well as any vector or recombinant nucleic acid (DNA, RNA, PNA, LNA, or any hybrid thereof; linear or circular; independent of strandedness) comprising such variable region nucleic acid. Any host cell comprising such variable region nucleic acid sequence, vector or recombinant nucleic acid is likewise part of the invention.

A further aspect of the invention relates to compounds capable of neutralizing HCV infection with said compounds comprising at least one CDR as described above or at least one variable region as described above. Such a compound can be used in passive immunization of a healthy or HCV infected mammal. Clearly, said passive immunization can be combined with any other HCV therapy or any other anti-HCV medicament, and wherein said combination occurs prior to, simultaneously with, or after said other HCV therapy or said other anti-HCV medicament.

Also, such a compound is applicable in methods for determining the neutralizing activity of a compound on HCV infection, said methods including use of said compound as a positive control compound for neutralization of HCV infection. Said compounds can also be incorporated in a composition further comprising for instance a carrier, adjuvant, or diluent. Examples of such compounds are protein aptamers, and bispecific antibodies or active fragments thereof.

The invention further relates to in vitro methods for identifying compounds capable of neutralizing HCV infection, said methods including the steps of:
(i) setting up an assay allowing an isolated CDR or isolated variable region as described above, or a compound comprising at least one CDR or at least one variable region as described above, to interact with E1, or with parts of E1 comprising SEQ ID NO:17,
(ii) adding the compound to be assessed for HCV neutralizing activity prior to, concurrently with, or after contacting an isolated CDR or isolated variable region as described above, or a compound comprising at least one CDR or at least one variable region as described above with E1 or parts of E1 as in as in (i),
(iii) reading out the binding of an isolated CDR or isolated variable region as described above, or a compound comprising at least one CDR or at least one variable region as described above, with said E1 or parts of E1,
(iv) identifying, from (iii), whether or not the compound added in (ii) qualifies as a compound capable of interfering with the interaction between an isolated CDR or isolated variable region as described above, or a compound comprising at least one CDR or at least one variable region as described above, and said E1 or parts of E1,
(v) confirming the neutralizing activity of the compound identified in (iv) in an HCV neutralization assay.

Any host cell comprising and/or secreting (i) a neutralizing anti-HCV antibody of the invention, (ii) an active fragment of (i), (iii) a CDR amino acid sequence of (i), (iv) a variable region amino acid sequence of (i), or (v) a compound comprising (i), (ii), (iii) or (iv) is likewise part of the invention.

### EXAMPLES

### Biological Deposits

The following hybridoma cell lines secreting monoclonal antibodies as mentioned throughout the specification were deposited in accordance with the Budapest Treaty:

| Hybridoma cell | | Deposit | |
|---|---|---|---|
| line | Deposit date | institution | Accession Number |
| IGH 388 | 13 Sep. 2000 | DSMZ | DSM ACC2470 |
| IGH 201 | 13 Mar. 1998 | ECACC | 98031216 |
| IGH 207 | 13 Mar. 1998 | ECACC | 98031214 |
| 17H1D9 (IGH 520) | 27 Sep. 2005 | DSMZ | DSM ACC 2734 |
| 48G5C4 (IGH 526) | 27 Sep. 2005 | DSMZ | DSM ACC 2736 |

The particulars of the deposit institutions are:
DSMZ: Deutsche Sammlung von Mikroorganismen und Zellkulturen GmbH, Mascheroder Weg 1b, D-38124 Braunschweig, Germany; and
ECACC: European Collection of Cell Cultures, Centre for Applied Microbiology and Research, Salisbury, Wiltshire, SP4 OJG, United Kingdom.

The notations “17H1”, “17H1D9”, and “IGH 520” are used interchangeably throughout the specification for the subject hybridoma cell line or the monoclonal antibody secreted by the hybridoma cell line.

The notations “48G5”, “48G5C4”, and “IGH 526” are used interchangeably throughout the specification for the subject hybridoma cell line or the monoclonal antibody secreted by the hybridoma cell line.

The notations “5D2”, “5D2H9” and “IGH 534” are used interchangeably throughout the specification for the subject hybridoma cell line or the monoclonal antibody secreted by the hybridoma cell line.

### Example 1

### Murine antibodies against E1

The generation and epitope mapping of monoclonal antibodies directed against E1 and used in the subsequent Examples have been described in WO 99/50301 (Examples 1 and 4 therein). More particularly the following antibodies were used. All these antibodies are of the IgG1 isotype.

| Name | recognizing epitope in E1 represented by aa region |
|---|---|
| IGH 200 | aa 212-226 |
| IGH 201 | aa 212-226 (ECACC accession number 98031216) |
| IGH 202 | aa 212-226 |
| IGH 204 | aa 212-226 |
| IGH 207 | aa 307-326 (ECACC accession number 98031214) |
| IGH 209 | aa 307-326 |
| IGH 210 | aa 307-326 |

The epitope region in E1 has been deduced from Example 4 in WO 99/50301, based on the smallest region common to all polypeptides reactive with the specific antibodies. In addition all antibodies recognize the E1s which covers the aa 192-326 of the HCV polyprotein.

### Example 2

### Human Antibodies Against E1

Fusion 1: The monoclonal hybridoma IGH 388 (DSMZ accession number DSM ACC2470) of which the antibody is recognizing an epitope within the E1 region aa 228-240 has been described in detail in European Patent Publication No. 1 574 517 (Examples 7 and 8 therein).
Fusion 2: Human volunteers were vaccinated with E1s. The details of this clinical phase I study have been described in Example 16 and 17 of WO 03/051912. PBMC of volunteer 003 and 004 were used to generate monoclonal antibodies with a procedure similar to the one as described for fusion 1. In brief, after sublethal irradiation, 2 NOD/SCID mice were injected i.p. with 1 mg of the anti-IL-2Rβ monoclonal antibody TMβ1. One day later the mice were injected i.s. with a mixture of 10⁷ PBMC and 5 μg E1s. The mice were injected i.p. with 10 μg E1s. Seven days later the mice were killed and the PBMC were isolated from the spleen. The number of cells that was recovered was 7×10⁶ and 6×10⁶ cells for respectively donor 003 and 004. FACS analysis of the cells showed that most of the cells were from human origin and that about 55 to 60% of the cells were B cells. The cells were fused with the K6H6/B5 hybridoma at a ratio of 1 spleen cell for 3 hybridomas, and were plated at 10⁴ splenic cells per well in DMEM/hyb supplemented with 20% Foetal Clone I serum, β-mercaptoethanol, aminopterin, IL-6, insulin like growth factor, gentamycin and ouabain.

Screening for antibodies specific to E1 was performed by a coating ELISA. In short: microtiter plates were coated overnight with 50 μl/well E1s at 0.5 μg/ml. The plates were washed once and blocked with PBS with 0.1% casein. Then the plates were incubated with supernatants from the hybridomas during 1 h. The human anti-E1 monoclonal antibody IGH 388 was used as positive control at 1 μg/ml. The plates were washed 4 times and incubated with a 1/2000 dilution in blocking buffer of HRP-conjugated sheep anti-human Ig (Amersham NA933) for 30 min at room temperature. The plates were washed 5 times and were incubated with TMB in HRP-substrate buffer for 30 minutes at room temperature. The reaction was stopped with H₂SO₄ and the O.D. values were read at 450-595 nm.

Nine wells had an O.D. value of more than 2.0 in the first screening. All these hybridomas were subcloned at 30, 10 and 3 cells per well. Finally, out of the nine initial hybridomas, 5 stable subclones were retained, these are subclones for the hybridomas: 3H2, 4G2, 7A2 and 12F3.

Subclass determination: 3H2 is of the IgG1 subclass, 4G2F12, 7A2B5, and 12F2C3 are of the IgM subclass.

Epitope mapping: Binding to the E1 peptides (IGP1036, 1022, 1177, 1176, 1039, 1549 and 898; see Table 1) was investigated. In short: microtiter plates are coated overnight with streptavidin (Roche) at 1 μg/ml, washed once and blocked with blocking buffer for 30 minutes. Then the following incubations are done: peptides at 100 ng/ml, supernatants of the hybridomas and HRP-conjugated sheep anti-human IgG (Amersham, 1/2000). 3H2 recognizes peptide IGP 898. 12F3 recognizes IGP 888. 4G2 and 7A2 don't bind to any of the peptides tested and are classified as recognizing a conformational epitope specific to E1.

Fusion 3 A human donor (2025) who had been previously infected with HCV but cleared the virus after IFN based therapy was randomly selected for generation of monoclonal antibodies with a procedure similar to the one as described for fusion 2. In brief, after sublethal irradiation, 1 NOD/SCID mouse was injected with 1 mg of the anti-IL-2Rβmonoclonal antibody TMβ1. One day later, the mouse was injected with 2×10⁷ PBMC from donor BB. The mouse was boosted with 5 μg E1s and E2s. Seven days later the mouse was killed, the spleen was removed and spleen cells were isolated. The number of cells that was recovered was 1.17×10⁷. FACS analysis of the cells showed that about 50% of the cells were from human origin and that about 35% of the cells were B cells (CD19 positive). All the cells were fused with SP2/0 Ag14 at a ratio of 3 myeloma cells per spleen cell. The cells were plated at 10³ spleen cells per well in DMEM/hyb supplemented with 20% Foetal Clone I serum, β-mercaptoethanol, aminopterin, IL-6, insulin like growth factor, gentamycin and ouabain.

Screening for antibodies specific to E1 was performed by a capture ELISA. In short: microtiter plates were coated overnight with goat anti-human IgG (H+L) (Jackson 109-005-088) at 0.9 μg/ml. The plates were washed once and blocked with PBS with 0.1% casein. Then the plates were incubated overnight with 100 μl supernatant of the hybridomas and 100 μl of blocking buffer supplemented with 0.4% Triton-X-705. The human monoclonal antibody IGH 388 was used as positive control. Then the plates were incubated with E1s, 10 ng/ml, followed by the biotinylated mouse anti-E1 monoclonal antibody IGH198 (a monoclonal derived from the same fusion as the antibodies described in Example 1). After washing, the plates were incubated for 30 minutes with HRP-conjugated streptavidin (Jackson, 100 ng/ml). Then the plates were washed 5 times and were incubated with TMB in HRP-substrate buffer for 30 minutes at room temperature. The reaction was stopped by acidification and the O.D. values were read at 450-595 nm.

Four antibodies specific to E1 were obtained and the hybridomas were subcloned at 30, 10, 3 and 1 cell per well. Finally, out of the four initial hybridomas, 3 stable subclones were retained, these are subclones for the hybridomas: 17H1 and 48G5 and 5D2.

Subclass determination: All antibodies are of the IgG1 subclass. 17H1 and 48G5 both have a lambda light chain and 5D2 a kappa light chain.
Epitope mapping: Binding to the E1 peptides (IGP 1036, 1022, 1177, 1176, 1039, 1549 and 898; see Table 1) was investigated. In short: microtiter plates are coated overnight with streptavidin (Roche) at 1 μg/ml, washed once and blocked with blocking buffer for 30 minutes. Then the following incubations are done: peptides at 100 ng/ml, supernatants of the hybridomas and HRP-conjugated sheep anti-human IgG (Amersham, 1/2000). A strong reaction against IGP 1176 and 1039 was seen with the monoclonal antibodies 17H1, 48G5 and 5D2.

In summary the following human monoclonal antibodies specific to E1 were derived from the 3 fusions:

| Name | recognizing epitope in E1 represented by aa region |
|---|---|
| IGH 388 | aa 228-240 |
| 3H2 | aa 313-326 |
| 4G2 | conformational |
| 7A2 | conformational |
| 12F3 | aa 192-228 |
| 17H1 | aa 307-326 |
| 48G5 | aa 307-326 |
| 5D2 | aa 307-326 |

The epitope region in E1 has been deduced from the smallest region common to all polypeptides reactive with the specific antibodies as described in the epitope mappings. In addition all antibodies recognize the E1s which covers the aa 192-326.

### Example 3

### Neutralizing Activity

Production and neutralization of retroviral pseudoparticles (pp) bearing HCV envelope glycoproteins. The pp were produced as described previously (Schofield et al., 2005 and Bartosch et al., 2003). All procedures were performed in the presence of 5-10% fetal calf serum. Test antibody samples were incubated for 1 h at room temperature with HCV pp, added to Huh-7 cells and incubated at 37° C. Supernatants were removed after 8 h and the cells were incubated in DMEM/10% FCS for 72 h at 37° C. GFP-positive cells were quantified by FACS analysis. The percent neutralization by each monoclonal antibody was calculated by comparison with results obtained in the absence of antibody. Neutralization titers were determined by serial two-fold dilutions of the monoclonal antibodies in DMEM, followed by incubation with the HCV pp. Neutralization was defined as ≧50% reduction of the number of GFP-positive cells.

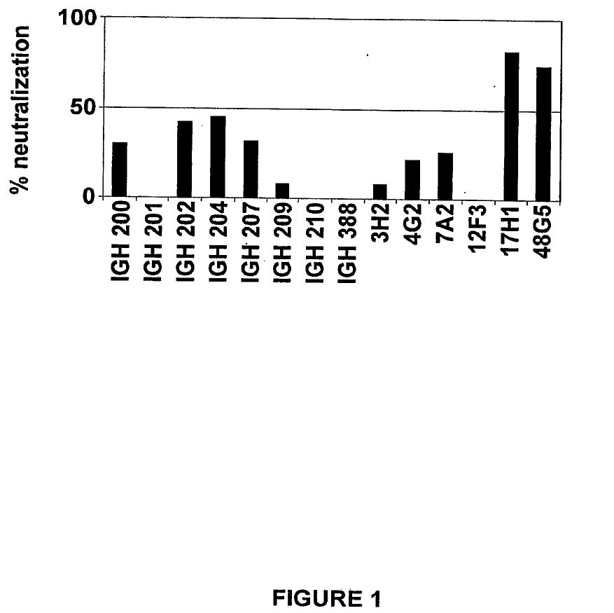

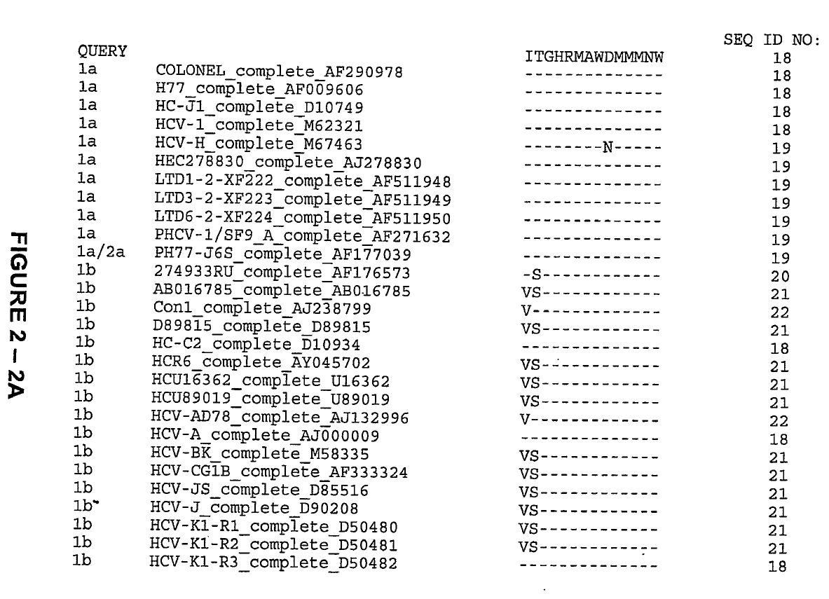

Preliminary Screen. Fourteen out of the 15 monoclonal antibodies of human or murine origin described in Examples 1 and 2 were tested for their ability to neutralize retroviral pseudoparticles bearing recombinant E1 and E2 glycoproteins derived from HCV genotype 1a strain H77. Only two of the monoclonal antibodies neutralized these prototype pseudoparticles (FIG. 1). These two monoclonal antibodies (17H1 and 48G5) are both derived from the same human donor and both recognize the same epitope near the C-terminal end of E1s. However, several other monoclonal antibodies recognizing the same epitope region (IGH 207, 209 and 210) or a part of this epitope region (3H2) were not neutralizing.

### Example 4

### Identification of Neutralizing Epitope Located at the C-Terminal End of E1s

As out of 6 monoclonal antibodies tested which recognize the aa region 307-326, only 2 were neutralizing, a more detailed epitope mapping for all monoclonal antibodies recognizing this region was performed. Human monoclonal antibodies against this region have also been previously described by Siemoneit et al. (1995). These authors studied the human immune response against the aa region 314-330. They identified 4 antibodies recognizing this region and mapped them by scanning using 9-mer peptides overlapping by 1 amino acid only. The two IgG antibodies could be mapped to the amino acid sequences RMAWDM (SEQ ID NO:46) and WDMMMNW (SEQ ID NO:47), respectively. Mapping of the two other antibodies of the IgM isotype was not clear cut which was attributed by the authors to their IgM nature.

In order to map the antibodies 17H1, 48G5, 5D2, 3H2, IGH207, IGH209 and IGH210 in more detail their reactivity was analyzed by means of ELISA. The previously used peptides IGP 898, 1176, 1039 and two novel peptides IGP2137 and 2138 (see Table 1) which represent the epitopes as identified by Siemoneit et al. (1995) were used for this epitope mapping. In short these biotinylated peptides were bound to streptavidin sensitized microtiterplates in a concentration of 5 μg/ml and allowed to react with the antibodies. Binding was detected using an anti-human PO labeled conjugate.

As shown in Table 2, this analysis allows more precise mapping of the epitopes for several antibodies. The epitope region in E1 has again been deduced based on the smallest region common to all polypeptides reactive with the specific antibodies. In addition all antibodies recognize the E1s which covers the amino acids 192-326.

| Name | recognizing epitope in E1 represented by aa region |
|---|---|
| 3H2 | aa 320-326 |
| 5D2 | aa 313-326 |
| 17H1 | aa 313-326 |
| 48G5 | aa 313-326 |
| IGH 207 | aa 313-326 |
| IGH 209 | aa 320-322 |
| IGH 210 | aa 320-326 |

TABLE 1

E1 peptides used for epitope mapping with indication
of the chemical modifications of the amino- and
carboxy-termini of the peptides and position (Start/End)
of the amino acids relative to the HCV polyprotein.

| | | | | | | SEQ ID |
|---|---|---|---|---|---|---|
| IGP | N-Terminal | AA Sequence | C-Terminal | Start | End | NO: |
| 888 | NH2- | YEVRNVSGIYHVTNDCSNSS | -GGK(Biotin)- | 192 | 228 | 38 |
| | | IVYEAADMIMHTPGC | CONH2 | | | |
| 898 | Biotin-GG | ITGHRMAWDMMMNWSPTAAL | —CONH2 | 313 | 332 | 39 |
| 1022 | Acetyl- | VRENNSSRCWVALTPTLAAR | Bio-K(peptide-G)- | 230 | 263 | 40 |
| | | NASVPTTTIRRHVD | GG-NH2 | | | |
| 1036 | Acetyl- | IVYEAADMIMHTPGCVPCVR | -G-K(Bio)-G-G | 212 | 241 | 41 |
| | | ENNSSRCWV | | | | |
| 1039 | Biotin-GG | SIYPGHITGHRMAWDMMMNW | —CONH2 | 307 | 340 | 42 |
| | | SPTTALVVSQLLRI | | | | |
| 1176 | Biotin- | SQLFTISPRRHETVQDCNCS | —CONH2 | 288 | 327 | 43 |
| | | IYPGHITGHRMAWDMMMNWS | | | | |
| 1177 | Biotin- | VALTPTLAARNASVPTTTIR | —CONH2 | 240 | 303 | 44 |
| | | RHVDSQLFTISPRRHETVQD | | | | |
| 1549 | Biotin- | IVYEAADMIMHTPGC | —CONH2 | 212 | 226 | 45 |
| 2137 | Biotin-GG | RMAWDM | —COOH | 317 | 322 | 46 |
| 2138 | Biotin-GG | WDMMMNW | —COOH | 320 | 326 | 47 |

The above analysis in fact revealed three groups of antibodies. The first group consisting of the antibody IGH 209 is recognizing a very small epitope represented by the amino acids 320-322 (WDM). The second group consisting of the antibodies 3H2 and IGH 210 recognizes a somewhat larger epitope represented by the amino acids (320-326). Finally the third group consisting of the antibodies 5D2, 17H1, 48G5 and IGH 207 recognizes an epitope located in a larger region represented by the amino acids 313-326. The human IgG antibodies previously described by Siemoneit et al. (1995) are similar to the group 1 and 2 antibodies identified here. Both neutralizing antibodies were found in the third group. In fact only human IgG antibodies were able to neutralize and not the murine derived antibody IGH 207 which did not have any neutralizing activity at 50 μg/ml as tested in Example 3. Alternatively only the human antibodies with a lambda light chain were able to neutralize (5D2 tested in Example 6). Note that the other antibodies of Siemoneit which could not be clearly mapped or both of the IgM isotype, so they are different.

TABLE 2
Reactivity of antibodies to peptides derived from the .C-terminal region of E1s. OD
values 2 time above the negative controls in the assay are considered
positive signals and have been marked in gray.

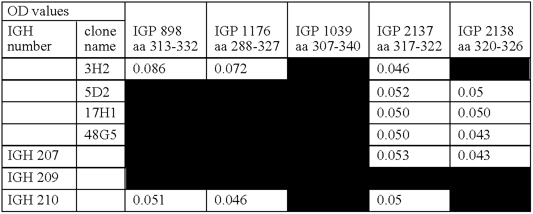

The region of amino acid 313-326, which is the region representing the neutralizable epitope is a well conserved region in E1 as shown in FIG. 2 which represents an alignment of this epitope to region performed on the HCV Los Alamos database (http://hcv.lanl.gov/content/hcv-db/index) on 5 Jan. 2006. Based on the alignment the sequence of this region is:

| | |
|---|---|
| X1-X2-G-X3-X4-MAW-X5-M-X6-X7-X8-W | (SEQ ID NO:17) |

wherein

### X1 is I, V, L or A;

### X2 is T or S;

### X3 is H or Q;

### X4 is R, H or K;

### X5 is D or N;

### X6 is M or I;

### X7 is M or L; and

### X8 is N, S or K.

### Example 5

### Cross-Neutralization

Production and neutralization of retroviral pseudoparticles (pp) bearing HCV envelope glycoproteins of other genotypes. To produce the pp of genotypes 2-6, we replaced the HCV sequence of phCMV-7a (Bartosch et al., 2003) with that of the 3′-terminal core and the entire E1 and E2 genes from HCV isolates representing the consensus sequence of the other genotypes (Meunier et al., 2005). For the 2a construct [pCMV-J6CF(2a)], the HCV sequence of the infectious clone pJ6CF was used (Yanagi et al., 1999). For the 3a [pCMV-S52(3a-11)], 4a [pCMV-ED43(4a-1)], 5a [pCMV-SA13(5a-12)], and 6a [pCMV-HK(6a-2.1)] constructs, the consensus sequence obtained from the acute phase chimpanzee plasma pools containing HCV strains S52, ED43, SA13, and HK-6a, respectively were used (Bukh et al., 1998; Bukh et al., 1993; Chamberlain et al., 1997). For the 1b construct, the E1 and E2 sequence of HCCl 66 was used (SEQ ID NO:49 of WO 1996/04385).

Results. The neutralization titer of the two neutralizing antibodies identified in Example 3 was determined against retroviral pseudotyped particles representing each of the six HCV genotypes. As summarized in Table 3 both antibodies neutralized genotype I a pseudotype particles. In this assay, antibody 48G5 was the most potent. Both antibodies weakly neutralized genotype 2a pp and were unreactive at the highest concentration tested against genotype 3a pp. In contrast, both antibodies relatively strongly neutralized genotype 4a, 5a and genotype 6a pp. The relative potency of the antibodies against the different pseudotypes varied.

Although similar cross-genotype neutralization has been previously reported for human polyclonal sera such as derived from patient H (Meunier et al., 2005) and other sera (Bartosch et al., 2003), it was so far not possible to obtain such cross-genotype neutralization with human monoclonal antibodies (recognizing E2) even if derived from the same patient H (Schofield et al., 2005). On the other hand neutralization across genotypes has been observed with a murine monoclonal directed against E2 (Owsianka et al., 2005). Neutralization with antibodies specific to E1 and more specifically cross-genotype neutralization with such antibodies has so far not been shown.

TABLE 3
Neutralization of antibodies 17H1 and 48G5 was tested in a dilution
series (2-fold dilution series) versus pp of genotypes 1 to 6. The data
represent the lowest concentration* of antibody in μg/ml exhibiting at
least 50% neutralization versus control samples.

| | Genotype | | | | | | |
|---|---|---|---|---|---|---|---|
| Hybridoma | 1a | 1b | 2a | 3a | 4a | 5a | 6a |
| 17H1 | 3.1 | 6.2 | 50 | >50 | <1 | <1 | 6.2 |
| 48G5 | <1 | 12.5 | 50 | >50 | 6.2 | 1.6 | 3.1 |

*final concentration in the assay; i.e., half of the concentration at the pre-incubation step - this explains the differences with Table 3 in U.S. Pat. No. 60/743,667 and EP06112063.0.

### Example 6

### Neutralizing Activity of 5D2

The neutralizing activity of 5D2 was not assessed in the screening of Example 3. This antibody recognizes an epitope in E1 similar to the antibodies 17H1 and 48G5. The neutralizing activity was assessed as described in Example 5 for 3 different genotypes: 1a, 2a and 4a. For genotype 2a, HCVpp were derived from the isolate JFH1 and for genotype 4a, HCVpp were derived from the isolated UKN4a. The results are presented in Table 4, and reveal a different cross-genotype neutralization profile compared to the antibodies 17H1 and 48G5 presented in Example 5. More particularly, the antibody 5D2 is more potently neutralizing genotype 2a than genotype 1a and 4a while the opposite is true for the antibodies 17H1 and 48G5 described in Example 5.

TABLE 4
Neutralization of antibody 5D2 was tested in a dilution series
(2-fold dilution series) versus pp of genotypes 1 to 6. The data
represent the lowest concentration of antibody in μg/ml
(final concentration in the assay, i.e. half of the concentration at the
pre-incubation step) exhibiting at least 50% neutralization versus
control samples.

| | Genotype | | | | | |
|---|---|---|---|---|---|---|
| Hybridoma | 1a | 2a | 3a | 4a | 5a | 6a |
| 5D2 | 25 | 6.2 | nd | >25 | nd | nd |

### Example 7

### Sequencing of antibody 5D2

From 5D2 the stable subclone 5D2H9 was selected for sequencing.

The heavy and light variable chains cDNA sequence of the monoclonal antibody were determined. For the heavy variable region, DNA sequence analysis on cloned fragments and subsequent alignment revealed a consensus sequence with only minor ambiguities and/or differences located mainly in framework regions. The consensus amino acid sequence is shown in FIG. 3. For the light variable region sequencing was directly performed on the PCR product. The amino acid sequence deduced thereof is shown in FIG. 4. Theoretically, predicted CDR loops are indicated (based on consensus sequence rules). The corresponding DNA sequences are SEQ ID NOs 9-14 (CDRs) and 15-16 (variable regions); see FIGS. 5 and 6.

Amino acid sequencing was also performed on purified antibody up to about amino acid 36 for VH and 30 for VL. This allowed confirmation of the amino acid sequence as deduced from DNA sequencing up to the first CDR and this both for the light and heavy chain.

### Example 8

### Affinity Measurement

Affinity of the neutralizing anti-HCV E1 envelope protein antibodies was measured using peptide IGP 2254 (ITGHRMAWDMMMNWS; SEQ ID NO:48). Association and dissociation of this peptide to immobilized antibody was measured using BIAcore.

TABLE 5
Affinity measurement of HCV neutralizing antibodies
for binding to IGP 2254.

| | ka × 10⁵ | kd × 10⁻⁴ | KD × 10⁻⁹ |
|---|---|---|---|
| | (M⁻¹ s⁻¹) | (s⁻¹) | (M) |
| 17H1D9 | 23.5 | 3.57 | 0.15 |
| 48G5C4 | 15.4 | 3.05 | 0.20 |
| 5D2H9 | 17.1 | 0.946 | 0.06 |

### Example 9

### Alanine Scan of E1-Epitope Recognized by Neutralizing Antibody 5D2

As IGP 2254 (SEQ ID NO:48, see Example 8) is the smallest peptide which is recognized very well by 5D2, an alanine-scan was performed on this sequence. Each amino-acid was replaced by alanine (or a glycine in case alanine was present in the IGP 2254 sequence). As for IGP 2254, each alanine (glycine) variant was synthesized with an N-terminal biotin and two additional glycine residues as spacer between the biotin moiety and the epitope.

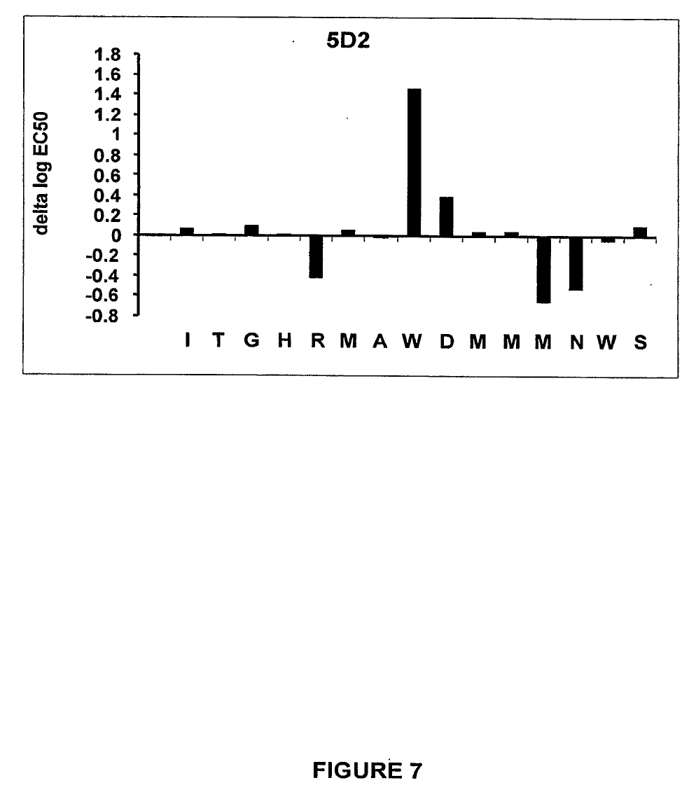

The binding of the antibody 5D2 was assessed in ELISA. In brief, biotinylated peptides are incubated on streptavidin coated plates. After washing, a serial dilution of the antibody is applied. Binding of antibodies to streptavidin bound peptide is detected by incubation with a secondary antibody specific for mouse immunoglobulines which is coupled to horse radish peroxidase. For each binding curve the EC50 is determined (antibody concentration at which half maximal binding is observed) using Prism software. In FIG. 7 the difference in log EC50 versus IGP 2254 for each of the alanine (glycine) variants is shown. A positive delta log EC50 indicates a reduced binding. A negative delta log EC50 indicates an increased binding.

### Example 10

### Natural Variants of E1-Epitope Recognized by Neutralizing Antibody 5D2

As IGP 2254 (SEQ ID NO:48, see Example 8) is the smallest peptide which is recognized very well by 5D2, a series of peptides was generated from the same region but representing natural variants. To search for natural variants the HCV sequence database (Kuiken C, Yusim K, Boykin L, Richardson R. The Los Alamos HCV Sequence-Database. Bioinformatics (2005), 21(3):379-84) was analyzed for known variants of this region. Each sequence occurring more than once in the database was finally synthesized as synthetic peptide with an N-terminal biotin and two additional glycine residues as spacer between the biotin moiety and the epitope.

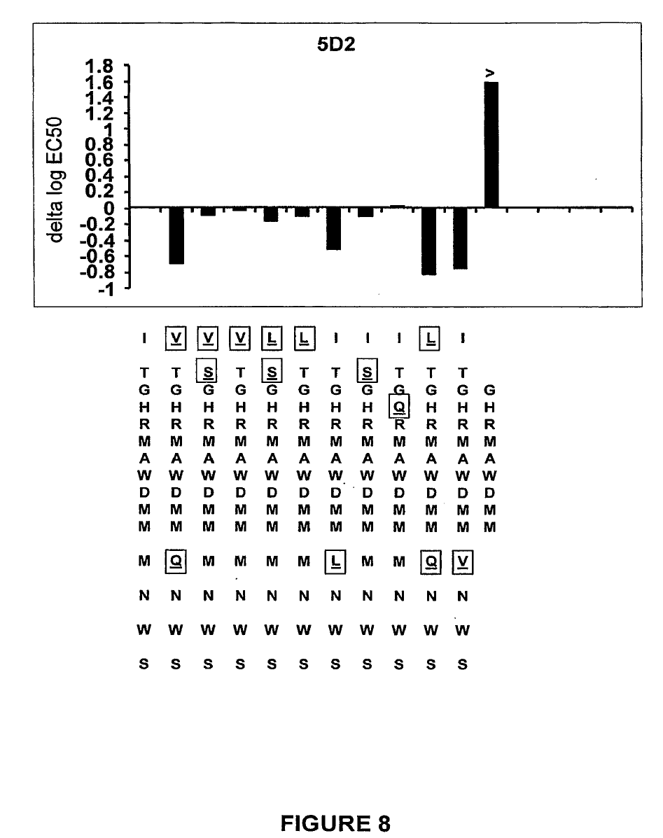

The binding of the antibody 5D2 was assessed in ELISA. In brief, biotinylated peptides are incubated on streptavidin coated plates. After washing, a serial dilution of the antibody is applied. Binding of antibodies to streptavidin bound peptide is detected by incubation with a secondary antibody specific for mouse immunoglobulines which is coupled to horse radish peroxidase. For each binding curve the EC50 is determined (antibody concentration at which half maximal binding is observed) using Prism software. In FIG. 8 the difference in log EC50 versus IGP 2254 for each of the natural sequence variants is shown. A positive delta log EC50 indicates a reduced binding. A negative delta log EC50 indicates an increased binding.

In addition, peptide IGP 3472 representing the very well conserved central region of the epitope (GHRAWDMM; SEQ ID NO:49) was also synthesized as synthetic peptide with an N-terminal biotin and two additional glycine residues as spacer between the biotin moiety. IGP 3472 was found not to be recognized by 5D2 as evidenced in FIG. 8.

### Example 11

### Further Detailed Epitope Analysis of Neutralizing Antibodies Recognizing the C-Terminal End of E1s

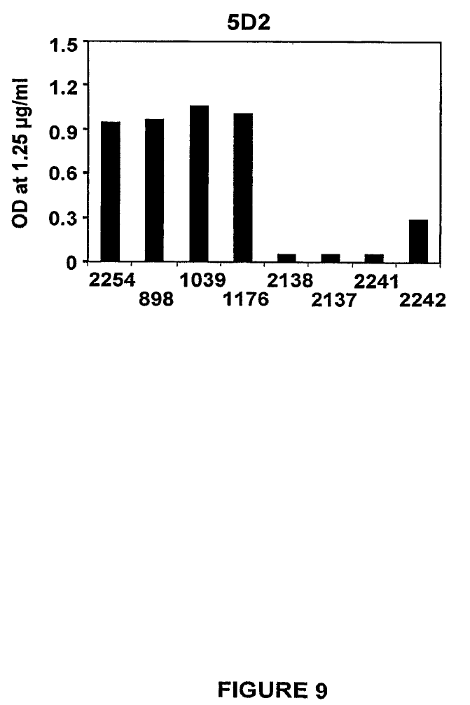

The epitopes of the neutralizing antibodies directed against E1 were mapped in greater detail using a series of peptides (see Table 6) including additional peptides compared to Example 4. As can be judged from FIG. 9 the neutralizing antibodies recognize different determinants in the epitope region 313-327.

The smallest peptide recognized by 48G5 is IGP 2254=E1 region 313-327. The minimal epitope can be further narrowed for this mAb to the region 313-326 as this antibody recognizes E1s which covers the amino acids 192-326.

The smallest peptide recognized by 17H1 is IGP 2241=E1 region 313-321.

The smallest peptide recognized by 5D2 is IGP 2242=region 321-330 but the reactivity is significantly lower than for the larger peptides such as IGP 2254 (aa 313-327). The minimal epitope can be further narrowed for this mAb to the region 321-326 as this antibody recognizes E1s which covers the amino acids 192-326. Nevertheless the peptide IGP 2138 (aa 320-326), which was recognized by the non-neutralizing antibody 3H2, is not recognized by this antibody. Consequently, important amino acids of the 5D2 epitope are to be found outside the region 321-326. The epitope recognized by 5D2 is thus best represented by the aa region 313-326.

TABLE 6

E1 peptides used for more detailed epitope mapping, with
indication of the chemical modifications of the amino-
and carboxy-termini of the peptides and position (Start/End)
of the amino acids relative to the HCV polyprotein.

| | | | | | | SEQ ID |
|---|---|---|---|---|---|---|
| IGP | N-Terminal | AA Sequence | C-Terminal | Start | End | NO: |
| 898 | Biotin-GG | ITGHRMAWDMMNNWSPTAAL | —CONH2 | 313 | 332 | 39 |
| 1036 | Acetyl- | IVYEAADMIMHTPGCVPCVR | -G-K(Bio)-G-G | 212 | 241 | 41 |
| | | ENNSSRCWV | | | | |
| 1039 | Biotin-GG | SIYPGHITGHRMAWDMMMNW | —CONH2 | 307 | 340 | 42 |
| | | SPTTALVVSQLLRI | | | | |
| 1176 | Biotin- | SQLFTISPRRHETVQDCNCS | —CONH2 | 288 | 327 | 43 |
| | | IYPGHITGHRMAWDMMMNWS | | | | |
| 2137 | Biotin-GG | RMAWDM | —COOH | 317 | 322 | 46 |
| 2138 | Biotin-GG | WDMMMNW | —COOH | 320 | 326 | 47 |
| 2241 | Biotin-GG | ITGHRMAWD | —CONH2 | 313 | 321 | 50 |
| 2242 | Biotin-GG | DMMMNWSPTA | —CONH2 | 321 | 330 | 51 |
| 2254 | Biotin-GG | ITGHRMAWDMMMNWS | —CONH2 | 313 | 327 | 48 |

### REFERENCE LIST

Allander, T., Drakenberg, K., Beyene, A., Rosa, D., Abrignani, S., Houghton, M., Widell, A., Grillner, L., and Persson, M. A. (2000) Recombinant human monoclonal antibodies against different conformational epitopes of the E2 envelope glycoprotein of hepatitis C virus that inhibit its interaction with CD81. J Gen. Virol. 81:2451-2459.
Barton, G. M. and Medzhitov, R. (2002) Toll-like receptors and their ligands. Curr. Top. Microbiol. Immunol. 270:81-92.
Bartosch, B., Bukh, J., Meunier, J. C., Granier, C., Engle, R. E., Blackwelder, W. C., Emerson, S. U., Cosset, F. L., and Purcell, R. H. (2003a) In vitro assay for neutralizing antibody to hepatitis C virus: evidence for broadly conserved neutralization epitopes. Proc. Natl. Acad. Sci. U.S.A. 100:14199-14204.
Bartosch, B., Dubuisson, J., and Cosset, F. L. (2003b) Infectious hepatitis C virus pseudo-particles containing functional E1-E2 envelope protein complexes. J Exp. Med 197:633-642.
Berke, J. M., Moradpour, D. (2005) Hepatitis C virus comes full circle: Production of recombinant infectious virus in tissue culture. Hepatology 42:1264-9.
Bukh, J., Apgar, C. L., Engle, R., Govindarajan, S., Hegerich, P. A., Tellier, R., Wong, D. C., Elkins, R., and Kew, M. C. (1998) Experimental infection of chimpanzees with hepatitis C virus of genotype 5a: genetic analysis of the virus and generation of a standardized challenge pool. J Infect. Dis. 178:1193-1197.
Bukh, J., Purcell, R. H., and Miller, R. H. (1993) At least 12 genotypes of hepatitis C virus predicted by sequence analysis of the putative E1 gene of isolates collected worldwide. Proc. Natl. Acad. Sci. U.S.A. 90:8234-8238.
Buonocore L., Blight, K. J., Rice, C. M., Rose, J. K. (2002) Characterization of vesicular stomatitis virus recombinants that express and incorporate high levels of hepatitis C virus glycoproteins. J. Virol. 76: 6865-6872.
Burioni, R., Plaisant, P., Manzin, A., Rosa, D., Delli Carri, V., Bugli, F., Solforosi, L., Abrignani, S., Varaldo, P. E., Fadda, G., Clementi, M. (1998) Dissection of human humoral immune response against hepatitis C virus E2 glycoprotein by repertoire cloning and generation of recombinant Fab fragments. Hepatology 28:810-814.
Byl, B., Libin, M., Bauer, J., Martin, O. R., De Wit, D., Davies, G., Goldman, M., and Willems, F. (2003) OM197-MP-AC induces the maturation of human dendritic cells and promotes a primary T cell response. Int Immunopharmacol. 3:417-425.
Chamberlain, R. W., Adams, N., Saeed, A. A., Simmonds, P., and Elliott, R. M. (1997) Complete nucleotide sequence of a type 4 hepatitis C virus variant, the predominant genotype in the Middle East. J Gen. Virol. 78 (Pt 6):1341-1347.
Falck-Ytter, Y., Kale, H., Mullen, K. D., Sarbah, S. A., Sorescu, L., and McCullough, A. J. (2002) Surprisingly small effect of antiviral treatment in patients with hepatitis C. Ann. Intern. Med. 136:288-292.
Farci, P., Shimoda, A., Wong, D., Cabezon, T., De Gioannis, D., Strazzera, A., Shimizu, Y., Shapiro, M., Alter, H. J., Purcell, R. H. (1996) Prevention of hepatitis C virus infection in chimpanzees by hyperimmune serum against the hypervariable region, 1 of the envelope 2 protein. Proc. Natl. Acad. Sci. U.S.A. 93:15394-15399.
Grakoui, A., Wychowski, C., Lin, C., Feinstone, S. M., and Rice, C. M. (1993) Expression and identification of hepatitis C virus polyprotein cleavage products. J. Virol. 67:1385-1395.
Habersetzer, F., Fournillier, A., Dubuisson, J., Rosa, D., Abrignani, S., Wychowski, C., Nakano, I., Trepo, C., Desgranges, C., and Inchauspe, G. (1998) Characterization of human monoclonal antibodies specific to the hepatitis C virus glycoprotein E2 with in vitro binding neutralization properties. Virology. 249:32-41.
Hsu, M., Zhang, J., Flint, M., Logvinoff, C., Cheng-Mayer, C., Rice, C. M., and McKeating, J. A. (2003) Hepatitis C virus glycoproteins mediate pH-dependent cell entry of pseudotyped retroviral particles. Proc. Natl. Acad. Sci. U.S.A. 100:7271-7276.
Johnson, D. A., Sowell, C. G., Johnson, C. L., Livesay, M. T., Keegan, D. S., Rhodes, M. J., Ulrich, J. T., Ward, J. R., Cantrell, J. L., and Brookshire, V. G. (1999) Synthesis and biological evaluation of a new class of vaccine adjuvants: aminoalkyl glucosaminide 4-phosphates (AGPs). Bioorg. Med. Chem. Lett 9:2273-2278.
Kabat, E. A., Wu, T. T., Perry, H. M., and Gottesman, K. S. F. C. (1991) Sequences of proteins of immunological interest, 5th edition. U.S. Department of Health and Human Services, Bethesda, Md.
Kaplan, M., Gawrieh, S., Cotler, S. J., and Jensen, D. M. (2003) Neutralizing antibodies in hepatitis C virus infection: a review of immunological and clinical characteristics. Gastroenterology. 125:597-604.
Keck, Z. Y., Op, D. B., Hadlock, K. G., Xia, J., Li, T. K., Dubuisson, J., and Foung, S. K. (2004a) Hepatitis C virus E2 has three immunogenic domains containing conformational epitopes with distinct properties and biological functions. J Virol. 78:9224-9232.
Keck, Z. Y., Sung, V. M., Perkins, S., Rowe, J., Paul, S., Liang, T. J., Lai, M. M., and Foung, S. K. (2004b) Human monoclonal antibody to hepatitis C virus E1 glycoprotein that blocks virus attachment and viral infectivity. J Virol. 78:7257-7263.
Klasse, P. J. and Sattentau, Q. J. (2002) Occupancy and mechanism in antibody-mediated neutralization of animal viruses. J Gen. Virol. 83:2091-2108.
Lagging, L. M., Meyer, K., Owens, R. J., and Ray, R. (1998) Functional role of hepatitis C virus chimeric glycoproteins in the infectivity of pseudotyped virus. J Virol. 72:3539-3546.
Lauer, G. M. and Walker, B. D. (2001) Hepatitis C virus infection. N. Engl J. Med. 345:41-52.
Li, C. and Allain, J. P. (2005) Chimeric monoclonal antibodies to hypervariable region 1 of hepatitis C virus. J Gen. Virol. 86:1709-1716.
Lindenbach, B. D., Evans, M. J., Syder, A. J., Wolk, B., Tellinghuisen, T. L., Liu, C. C., Maruyama, T., Hynes, R. O., Burton, D. R., McKeating, J. A., Rice, C. M. (2005) Complete replication of hepatitis C virus in cell culture. Science 309:623-6.
Logvinoff, C., Major, M. E., Oldach, D., Heyward, S., Talal, A., Balfe, P., Feinstone, S. M., Alter, H., Rice, C. M., and McKeating, J. A. (2004) Neutralizing antibody response during acute and chronic hepatitis C virus infection. Proc. Natl. Acad. Sci. U.S.A. 101:10149-10154.
Manns, M. P., McHutchison, J. G., Gordon, S. C., Rustgi, V. K., Shiffman, M., Reindollar, R., Goodman, Z. D., Koury, K., Ling, M., and Albrecht, J. K. (2001) Peginterferon alfa-2b plus ribavirin compared with interferon alfa-2b plus ribavirin for initial treatment of chronic hepatitis C: a randomised trial. Lancet 358:958-965.
Meunier, J. C., Engle, R. E., Faulk, K., Zhao, M., Bartosch, B., Alter, H., Emerson, S. U., Cosset, F. L., Purcell, R. H., and Bukh, J. (2005) Evidence for cross-genotype neutralization of hepatitis C virus pseudo-particles and enhancement of infectivity by apolipoprotein C1. Proc. Natl. Acad. Sci. U.S.A. 102:4560-4565.
Owsianka, A., Tarr, A. W., Juttla, V. S., Lavillette, D., Bartosch, B., Cosset, F. L., Ball, J. K., and Patel, A. H. (2005) Monoclonal antibody AP33 defines a broadly neutralizing epitope on the hepatitis C virus E2 envelope glycoprotein. J Virol. 79:11095-11104.
Persing, D., Coler, R., Lacy, M., Johnson, D., Baldridge, J., Hershberg, R., and Reed, S. (2002) Taking toll: lipid A mimetics as adjuvants and immunomodulators. Trends Microbiol. 10:S32.
Riedl, P., Buschle, M., Reimann, J., and Schirmbeck, R. (2002) Binding immune-stimulating oligonucleotides to cationic peptides from viral core antigen enhances their potency as adjuvants. Eur. J. Immunol. 32:1709-1716.
Rosa, D., Campagnoli, S., Moretto, C., Guenzi, E., Cousens, L., Chin, M., Dong, C., Weiner, A. J., Lau, J. Y., Choo, Q. L., Chien, D., Pileri, P., Houghton, M., and Abrignani, S. (1996) A quantitative test to estimate neutralizing antibodies to the hepatitis C virus: cytofluorimetric assessment of envelope glycoprotein 2 binding to target cells. Proc. Natl. Acad. Sci. U.S.A. 93:1759-1763.
Schofield, D. J., Bartosch, B., Shimizu, Y. K., Allander, T., Alter, H. J., Emerson, S. U., Cosset, F. L., and Purcell, R. H. (2005) Human monoclonal antibodies that react with the E2 glycoprotein of hepatitis C virus and possess neutralizing activity. Hepatology 42:1055-1062.
Shiffman, M. L. (1999) Improvement in liver histopathology associated with interferon therapy in patients with chronic hepatitis C. Viral Hepatitis Reviews 5:27-43.
Shimizu, Y. K., Hijikata, M., Iwamoto, A., Alter, H. J., Purcell, R. H., and Yoshikura, H. (1994) Neutralizing antibodies against hepatitis C virus and the emergence of neutralization escape mutant viruses. J Virol. 68:1494-1500.
Shimotohno, K., Tanji, Y., Hirowatari, Y., Komoda, Y., Kato, N., and Hijikata, M. (1995) Processing of the hepatitis C virus precursor protein. J. Hepatol. 22:87-92.
Siemoneit, K., Cardoso, M. S., Koerner, K., Wolpl, A., and Kubanek, B. (1995) Human monoclonal antibodies for the immunological characterization of a highly conserved protein domain of the hepatitis C virus glycoprotein E1. Clin. Exp. Immunol. 101:278-283.
Simmonds, P., Bukh, J., Combet, C., Deleage, G., Enomoto, N., Feinstone, S., Halfon, P., Inchauspe, G., Kuiken, C., Maertens, G., Mizokami, M., Murphy, D. G., Okamoto, H., Pawlotsky, J. M., Penin, F., Sablon, E., Shin, I., Stuyver, L. J., Thiel, H. J., Viazov, S., Weiner, A. J., and Widell, A. (2005) Consensus proposals for a unified system of nomenclature of hepatitis C virus genotypes. Hepatology 42:962-973.
Triyatni, M., Saunier, B., Maruvada, P., Davis, A. R., Ulianich, L., Heller, T., Patel, A., Kohn, L. D., Liang, T. J. (2002) Interaction of hepatitis C virus-like particles and cells: a model system for studying viral binding and entry. J. Virol. 76:9335-9344.
Wakita, T., Pietschmann, T., Kato, T., Date, T., Miyamoto, M., Zhao, Z., Murthy, K., Habermann, A., Krausslich, H. G., Mizokami, M., Bartenschlager, R., Liang, T. J. (2005) Production of infectious hepatitis C virus in tissue culture from a cloned viral genome. Nat Med 11:791-6.
Walewski, J. L., Keller, T. R., Stump, D. D., and Branch, A. D. (2001) Evidence for a new hepatitis C virus antigen encoded in an overlapping reading frame. RNA. 7:710-721.
Winter, G. and Harris, W. J. (1993) Humanized antibodies. Immunol. Today 14:243-246.
Xu, Z., Choi, J., Yen, T. S., Lu, W., Strohecker, A., Govindarajan, S., Chien, D., Selby, M. J., and Ou, J. (2001) Synthesis of a novel hepatitis C virus protein by ribosomal frameshift. EMBO J. 20:3840-3848.
Yanagi, M., Purcell, R. H., Emerson, S. U., and Bukh, J. (1999) Hepatitis C virus: an infectious molecular clone of a second major genotype (2a) and lack of viability of intertypic 1a and 2a chimeras. Virology. 262:250-263.

1. An isolated anti-HCV E1 envelope protein antibody characterized in that said antibody is capable of neutralizing HCV infection.

2. The antibody according to claim 1 further characterized in that it comprises at least one of the complementarity determining regions (CDR) with an amino acid sequence chosen from SEQ ID NOs: 1 to 6 or a CDR with an amino acid sequence that is at least 80% identical with any of SEQ ID NOs: 1 to 6.

3. The antibody according to claim 1 further characterized in that it comprises a variable region with an amino acid sequence chosen from SEQ ID NOs: 7 or 8 or a variable region with an amino acid sequence that is at least 70% identical with any of SEQ ID NOs: 7 or 8.

4. The antibody according to claim 1 further characterized in that it comprises
a CDR triplet H1/H2/H3 or a CDR triplet that is at least 85% identical therewith; or
a CDR triplet L1/L2/L3 or a CDR triplet that is at least 85% identical therewith;
wherein H1 has the amino acid sequence of SEQ ID NOs: 1, H2 has the amino acid sequence of SEQ ID NOs: 2, H3 has the amino acid sequence of SEQ ID NOs: 3, L1 has the amino acid sequence of SEQ ID NOs: 4, L2 has the amino acid sequence of SEQ ID NOs: 5, and L3 has the amino acid sequence of SEQ ID NOs: 6.

5. The antibody according to claim 1 further characterized in that it is specific for binding an HCV E1 envelope protein epitope with SEQ ID NO: 17.

6. The antibody according to claim 1 further characterized in that it is a human monoclonal antibody or a humanized monoclonal antibody.

7. An active fragment of the antibody according to any claim 1 characterized in that said fragment is capable of neutralizing HCV infection.

8. A composition comprising the antibody according to claim 1, or the active fragment and at least one of a carrier, adjuvant, or diluent.

9. The composition according to claim 9 which is a vaccine composition.

10. A diagnostic kit for detecting HCV E1 antigens in a biological sample, said kit comprising the antibody according to claim 1, or the active fragment.

11. A method of producing the antibody according to claim 1 or active fragment thereof comprising the steps of:
(i) obtaining a crude preparation of said antibody or antibody fragment by means of recombinant expression of the antibody or antibody fragment, or by means of chemical synthesis of the antibody or antibody fragment;
(ii) purifying said antibody or antibody fragment from the crude preparation obtained in (i).

12. A method of producing the active fragment of the antibody according to claim 7 comprising the steps of:
(i) obtaining a crude preparation of an antibody comprising said fragment by means of recombinant expression of the antibody or by means of chemical synthesis of the antibody;
(ii) purifying said antibody from the crude preparation obtained in (i).
(iii) isolating the active fragment from the antibody purified in (ii).

13. The antibody according to claim 1, or the active fragment for use in passive immunization of a healthy or HCV infected mammal.

14. The antibody according to claim 1, or the active fragment for use in prevention of HCV recurrence in a non-HCV infected liver transplanted to a chronic HCV patient.

15. The antibody according to claim 1, or the active fragment for use in prevention of HCV infection in a non-HCV infected mammal.

16. The antibody according to claim 1, or the active fragment for use in prevention of HCV infection in a non-HCV infected mammal after an accident with an HCV-bearing needle-stick.

17. The antibody according to claim 1, or the active fragment for use in prevention of transmission of HCV infection during pregnancy and/or birth from an HCV infected mother mammal to its child.

18. The antibody according to claim 1, or the active fragment for use in treatment of HCV infection in an HCV infected mammal.

19. The antibody or fragment according to claim 13 wherein said passive immunization is combined with any other anti-HCV medicament or any other HCV therapy and wherein said combination occurs prior to, simultaneously with or after said other anti-HCV medicament or HCV therapy.

20. The antibody or fragment according to claim 14 wherein said medicament is combined with any other anti-HCV medicament and wherein said combination occurs prior to, simultaneously with or after said other anti-HCV medicament.

21. The antibody or fragment according to claim 18 wherein said medicament is combined with any other HCV therapy and wherein said combination occurs prior to, simultaneously with or after said other HCV therapy.

22. An in vitro method for identifying compounds capable of neutralizing HCV infection, said method including the steps of:
(i) setting up an assay allowing the antibody according to claim 1, or the active fragment to interact with E1, or with parts of E1 comprising SEQ ID NO:17,
(ii) adding the compound to be assessed for HCV neutralizing activity prior to, concurrently with, or after contacting the antibody with E1 or parts of E1 as in (i),
(iii) reading out the binding of the antibody with said E1 or parts of E1,
(iv) identifying, from (iii), whether or not the compound added in (ii) qualifies as a compound capable of interfering with the antibody-E1 interaction
(v) confirming the neutralizing activity of the compound identified in (iv) in an HCV neutralization assay.

23. A method for determining the neutralizing activity of a compound on HCV infection, said method including use of the antibody according to claim 1, or the active fragment as a positive control compound for neutralization of HCV infection.

24. The antibody or fragment according to claim 13 wherein said mammal is a human.

25. An isolated complementarity determining region (CDR) of an anti-HCV E1 envelope protein antibody capable of neutralizing HCV infection.

26. The CDR according to claim 25 which has an amino acid sequence chosen from SEQ ID NOs: 1 to 6 or an amino acid sequence that is at least 80% identical with any of SEQ ID NOs: 1 to 6.

27. The CDR according to claim 25 which is encoded by a nucleic acid sequence chosen from SEQ ID NOs: 9 to 14.

28. An isolated variable region of an anti-HCV E1 envelope protein antibody capable of neutralizing HCV infection.

29. The variable region according to claim 28 which has an amino acid sequence chosen from SEQ ID NOs: 7 or 8, or an amino acid sequence that is at least 70% identical with any of SEQ ID NOs: 7 or 8.

30. The variable region according to claim 29 which is encoded by a nucleic acid sequence chosen from SEQ ID NOs: 15 or 16.

31. A compound capable of neutralizing HCV infection comprising at least one CDR according to claim 25 or at least one variable region.

32. The compound according to claim 31 for use in passive immunization of a healthy or HCV infected mammal.

33. The compound according to claim 32 wherein said passive immunization is combined with any other HCV therapy or any other anti-HCV medicament, and wherein said combination occurs prior to, simultaneously with, or after said other HCV therapy or said other anti-HCV medicament.

34. An in vitro method for identifying compounds capable of neutralizing HCV infection, said method including the steps of:
(i) setting up an assay allowing the compound according to claim 31 to interact with E1, or with parts of E1 comprising SEQ ID NO:17,
(ii) adding the compound to be assessed for HCV neutralizing activity prior to, concurrently with, or after contacting the compound with E1 or parts of E1 as in (i),
(iii) reading out the binding of the compound with said E1 or parts of E1,
(iv) identifying, from (iii), whether or not the compound added in (ii) qualifies as a compound capable of interfering with the interaction between the compound and said E1 or part of E1,
(v) confirming the neutralizing activity of the compound identified in (iv) in an HCV neutralization assay.

35. A method for determining the neutralizing activity of a compound on HCV infection, said method including use of the compound according to claim 31 as a positive control compound for neutralization of HCV infection.

36. The compound according to claim 32 wherein said mammal is a human.

37. A composition comprising at least one CDR according to claim 25, and at least one of a carrier, adjuvant, or diluent.

38. A composition comprising at least one variable region according to claim 28, and at least one of an excipient, diluent or adjuvant.

39. A composition comprising a compound according to claim 31 and at least one of a carrier, adjuvant, or diluent.


SEQUENCE LISTING (51 sequences)

SEQ ID NO: 1 - protein, 10 aa, human
GFTFSHYAMH

SEQ ID NO: 2 - protein, 17 aa, human
VVSYDGSNKFYVDSVKG

SEQ ID NO: 3 - protein, 18 aa, human
DPGVVTGSWSDSDGPPIH

SEQ ID NO: 4 - protein, 17 aa, human
KSSQSVFYSSNNKNYLG

SEQ ID NO: 5 - protein, 7 aa, human
WASTRAS

SEQ ID NO: 6 - protein, 8 aa, human
QQYYTSWA

SEQ ID NO: 7 - protein, 127 aa, human
QVQLVESGGGVVQPGRSLRLSCVASGFTFSHYAMHWIRQAPGKGLEWVAVVSYDGSNKFYVDSVKGRFTISRDNSKNTVYLQMNSLRAVDTAVYYCARDPGVVTGSWSDSDGPPIHWGQGTLVTVSS

SEQ ID NO: 8 - protein, 113 aa, human
DIVVTQSPDSLAVSLGERATITCKSSQSVFYSSNNKNYLGWYQQKPGQPPKLLIYWASTRASGVPDRFSGSGSGTDFTLTISSLQAEDVAVYYCQQYYTSWAFGQGTKVEIKR

SEQ ID NO: 9 - DNA, 30 bp, human
ggattcaccttcagtcactatgcaatgcac

SEQ ID NO: 10 - DNA, 51 bp, human
gttgtctcatatgatggaagcaataaattctacgtagactccgtgaagggc

SEQ ID NO: 11 - DNA, 54 bp, human
gatcccggggtagtaacgggatcttggtctgacagtgatggtcctccgatacac

SEQ ID NO: 12 - DNA, 51 bp, human
aagtccagccagagtgttttctacagttccaacaataagaactacttaggt

SEQ ID NO: 13 - DNA, 21 bp, human
tgggcatctacccgggcatcc

SEQ ID NO: 14 - DNA, 24 bp, human
cagcaatattatacttcttgggcg

SEQ ID NO: 15 - DNA, 381 bp, human
caggtgcagctggtggagtctgggggaggcgtggtccagcctgggaggtccctgagactctcctgtgtggcctctggattcaccttcagtcactatgcaatgcactggatccgccaggctccaggcaaggggctggagtgggtggctgttgtctcatatgatggaagcaataaattctacgtagactccgtgaagggccgattcaccatctccagagacaactccaagaacacagtgtatttgcaaatgaatagcctgagagctgtagacacggccgtgtattactgtgcgagagatcccggggtagtaacgggatcttggtctgacagtgatggtcctccgatacactggggccagggaaccctcgtcaccgtctcctca

SEQ ID NO: 16 - DNA, 339 bp, human
gacatcgtggtgacccagtctccagactccctggctgtttctctgggcgagagggccaccatcacctgcaagtccagccagagtgttttctacagttccaacaataagaactacttaggttggtaccagcagaaaccgggacagcctcctaagctgctcatttactgggcatctacccgggcatccggggtccctgaccgattcagtggcagcgggtctgggacagatttcactctcaccatcagcagcctgcaggctgaagatgtggctgtttattactgtcagcaatattatacttcttgggcgttcggccaagggaccaaggtggaaatcaaacga

SEQ ID NO: 17 - protein, 14 aa, hepatitis C virus
  MISC_FEATURE (1)..(1): X = I, V, L or A
XXGXXMAWXMXXXW

SEQ ID NO: 18 - protein, 14 aa, hepatitis C virus
ITGHRMAWDMMMNW

SEQ ID NO: 19 - protein, 14 aa, hepatitis C virus
ITGHRMAWNMMMNW

SEQ ID NO: 20 - protein, 14 aa, hepatitis C virus
ISGHRMAWDMMMNW

SEQ ID NO: 21 - protein, 14 aa, hepatitis C virus
VSGHRMAWDMMMNW

SEQ ID NO: 22 - protein, 14 aa, hepatitis C virus
VTGHRMAWDMMMNW

SEQ ID NO: 23 - protein, 14 aa, hepatitis C virus
LTGHRMAWDMMMNW

SEQ ID NO: 24 - protein, 14 aa, hepatitis C virus
ASGHRMAWDMMMNW

SEQ ID NO: 25 - protein, 14 aa, hepatitis C virus
VSGHRMAWDMIMNW

SEQ ID NO: 26 - protein, 14 aa, hepatitis C virus
LSGHRMAWDMMMNW

SEQ ID NO: 27 - protein, 14 aa, hepatitis C virus
  MISC_FEATURE (2)..(2): X = any amino acid
IXGHRMAWDMMMNW

SEQ ID NO: 28 - protein, 14 aa, hepatitis C virus
  MISC_FEATURE (1)..(1): X = any amino acid
XTGHRMAWDMMMNW

SEQ ID NO: 29 - protein, 14 aa, hepatitis C virus
ITGQRMAWDMMMNW

SEQ ID NO: 30 - protein, 14 aa, hepatitis C virus
ITGHRMAWDMMLSW

SEQ ID NO: 31 - protein, 14 aa, hepatitis C virus
ITGQRMAWDMMLNW

SEQ ID NO: 32 - protein, 14 aa, hepatitis C virus
ITGHRMAWDMMLNW

SEQ ID NO: 33 - protein, 14 aa, hepatitis C virus
ITGHHMAWDMMLNW

SEQ ID NO: 34 - protein, 14 aa, hepatitis C virus
VTGQRMAWDMMLNW

SEQ ID NO: 35 - protein, 14 aa, hepatitis C virus
LSGQRMAWDMMMNW

SEQ ID NO: 36 - protein, 14 aa, hepatitis C virus
ITGHRMAWDMMMKW

SEQ ID NO: 37 - protein, 14 aa, hepatitis C virus
VTGHKMAWDMMMNW

SEQ ID NO: 38 - protein, 35 aa, hepatitis C virus
YEVRNVSGIYHVTNDCSNSSIVYEAADMIMHTPGC

SEQ ID NO: 39 - protein, 20 aa, hepatitis C virus
ITGHRMAWDMMMNWSPTAAL

SEQ ID NO: 40 - protein, 34 aa, hepatitis C virus
VRENNSSRCWVALTPTLAARNASVPTTTIRRHVD

SEQ ID NO: 41 - protein, 29 aa, hepatitis C virus
IVYEAADMIMHTPGCVPCVRENNSSRCWV

SEQ ID NO: 42 - protein, 34 aa, hepatitis C virus
SIYPGHITGHRMAWDMMMNWSPTTALVVSQLLRI

SEQ ID NO: 43 - protein, 40 aa, hepatitis C virus
SQLFTISPRRHETVQDCNCSIYPGHITGHRMAWDMMMNWS

SEQ ID NO: 44 - protein, 40 aa, hepatitis C virus
VALTPTLAARNASVPTTTIRRHVDSQLFTISPRRHETVQD

SEQ ID NO: 45 - protein, 15 aa, hepatitis C virus
IVYEAADMIMHTPGC

SEQ ID NO: 46 - protein, 6 aa, hepatitis C virus
RMAWDM

SEQ ID NO: 47 - protein, 7 aa, hepatitis C virus
WDMMMNW

SEQ ID NO: 48 - protein, 15 aa, hepatitis C virus
ITGHRMAWDMMMNWS

SEQ ID NO: 49 - protein, 9 aa, hepatitis C virus
GHRMAWDMM

SEQ ID NO: 50 - protein, 9 aa, hepatitis C virus
ITGHRMAWD

SEQ ID NO: 51 - protein, 10 aa, hepatitis C virus
DMMMNWSPTA